(19) United States
(12) Patent Application Publication
(10) Pub. No.: US 2003/0157070 A1
(43) Pub. Date: Aug. 21, 2003

(54) High efficiency ex vivo transduction of cells by high titer recombinant retroviral preparations

(76) Inventor: Douglas J. Jolly, Leucadia, CA (US)

Correspondence Address: Chiron Corporation, Intellectual Property - R440, P.O. Box 8097, Emeryville, CA, 94662-8097, US

(21) Appl. No.: 10/001,729

(22) Filed: Oct. 22, 2001

Related U.S. Application Data
(63) Continuation of application No. 09/191,448, filed on Nov. 12, 1998, now abandoned.
Application No. 09/191,448 is a continuation-in-part of application No. 08/425,180, filed on Apr. 20, 1995, now abandoned.
Application No. 08/425,180 is a continuation-in-part of application No. 08/367,071, filed on Dec. 30, 1994, now abandoned.

(51) Int. Cl. A61K 48/00; C12N 15/867; C12N 5/08

(52) U.S. Cl. 424/93.21; 435/455; 435/456; 435/372

(57) ABSTRACT

Compositions and methods for the efficient ex vivo introduction of nucleic acid into T cells, non-dividing cells, and cells resistant to standard transduction techniques mediated by high titer recombinant retroviral preparations is described. The recombinant vector constructs carried by the recombinant retrovirus particles code for the production of one or more desired gene products from one or more correponding genes of interest, at least one of the gene products having a therapeutic application. Upon re-introduction into a patient, the transduced cells produce a desired gene product in an amount sufficient to treat a particular disease state.

### CROSS-REFERENCE TO RELATED APPLICATIONS

This application is a continuation-in-part of co-pending U.S. application Ser. No. 08/425,180, filed Apr. 20, 1995, and a continuation-in part of co-pending U.S. application Ser. No. 08/367,071, filed Dec. 30, 1994, both of which are hereby incorporated by reference.

### TECHNICAL FIELD

The present invention relates generally to recombinant retroviruses and gene therapy, and more specifically, to high titer recombinant retroviral particle preparations suitable for a variety of gene therapy applications.

### BACKGROUND OF THE INVENTION

Since the discovery of DNA in the 1940s and continuing through the most recent era of recombinant DNA technology, substantial research has been undertaken in order to realize the possibility that the course of disease may be affected through interaction with the nucleic acids of living organisms. Most recently, a wide variety of methods have been described for altering or affecting genes of somatic tissue (a process sometimes referred to as “somatic gene therapy”), including for example, viral vectors derived from retroviruses, adenoviruses, vaccinia viruses, herpes viruses, and adeno-associated viruses (see Jolly, Cancer Gene Therapy l (1):51-64, 1994), as well as direct transfer techniques such as lipofection (Feigner et al., Proc. Natl. Acad. Sci. USA 84:7413-7417, 1989), direct DNA injection (Acsadi et al., Nature 352:815-818, 1991), microprojectile bombardment (Williams et al., PNAS 88:2726-2730, 1991), liposomes of several types (see, e.g., Wang et al., PNAS 84:7851-7855, 1987) and administration of nucleic acids alone (WO 90/11092).

Of these techniques, recombinant retroviral gene delivery methods have been most extensively utilized, in part due to: (1) the efficient entry, of genetic material (the vector genome) into replicating cells; (2) an active, efficient process of entry into the target cell nucleus; (3) relatively high levels of gene expression; (4) the potential to target particular cellular subtypes through control of the vector-target cell binding and the tissue-specific control of gene expression; (5) a general lack of pre-existing host immunity, and (6) substantial knowledge and clinical experience which has been gained with such vectors.

Briefly, retroviruses are diploid positive-strand RNA viruses that replicate through an integrated DNA intermediate. In particular, upon infection by the RNA virus, the retroviral genome is reverse-transcribed into DNA by a virally encoded reverse transcriptase that is carried as a protein in each retrovirus. The viral DNA is then integrated pseudo-randomly into the host cell genome of the infecting cell, forming a “provirus” which is inherited by daughter cells.

Wild-type retroviral genomes (and their proviral copies) contain the genes (the gag, pol and env genes), which are preceded by a packaging signal (Ψ), and two long terminal repeat (LTR) sequences which flank both ends. Briefly, the gag gene encodes the internal structural (nuleocapsid) proteins. The pol gene codes for the RNA-dependent DNA polymerase which reverse transcribes the RNA genome, and the env gene encodes the retroviral envelope glycoproteins. The 5′ and 3′ LTRs contain cis-acting elements necessary to promote transcription and polyadenylation of retroviral RNA.

Adjacent to the 5′ LTR are sequences necessary for reverse transcription of the genome (the tRNA primer binding site) and for efficient encapsidation of retroviral RNA into particles (the y sequence). Removal of the packaging signal prevents encapsidation (packaging of retroviral RNA into infectious virions) of genomic RNA, although the resulting mutant can still direct synthesis of all proteins encoded in the viral genome.

Recombinant retroviruses and various uses thereof have been described in numerous references including, for example, Mann, et al. (Cell 33:153, 1983), Cane and Mulligan (Proc. Nat'l. Acad. Sci. USA 81:6349, 1984), Miller, et al., Human Gene Therapy 1:5-14, 1990, U.S. Pat. Nos. 4,405,712; 4,861,719; 4,980,289 and PCT Application Nos. WO 89/02,468; WO 89/05,349 and WO 90/02,806). Briefly, a foreign gene of interest may be incorporated into the retrovirus in place of a portion of the normal retroviral RNA. When the retrovirus injects its RNA into a cell, the foreign gene is also introduced into the cell, and may then be integrated into the host's cellular DNA as if it were the retrovirus itself. Expression of this foreign gene within the host results in expression of the foreign protein by the host cell.

One disadvantage, however, of recombinant retroviruses is that the principally infect only replicating cells, thereby making efficient direct gene transfer difficult or impossible for cells characterized as largely non-replicating. In addition, several other types of cells including T cells, B cells, monocytic cells and dendritic cells have traditionally been difficult to transduce by retroviral vectors, even when stimulated to replicate. This was particularly true for primary cells. Indeed, some scientists have suggested that other, more efficient methods of gene transfer, such as direct administration of pure plasmid DNA, be utilized (Davis et al., Human Gene Therapy 4:733-740, 1993) to introduce nucleic acid molecules into such cells.

In order to increase the efficacy of recombinant retroviruses, the methods which have been suggested have principally been aimed at inducing the desired target cells to replicate or to replicate more efficiently, thereby allowing the retroviruses to infect the cells. Such methods have included, for example chemical treatment with 10% carbon tetrachloride in mineral oil (Kaleko, et al., Human Gene Therapy 2:27-32, 1991). However, such techniques are not preferred for use in ex vivo techniques designed to introduce nucleic acid molecules encoding therapeutic gene products into animal cells. For T cells, various methods of in vitro non-specific stimulation (such as IL-2, pokeweed mitogen, and anti-CD3 antibodies) can be quite efficient in inducing replication. However, difficulties remained in obtaining efficient transduction with methods compatible with clinical and commercial use.

Efficient gene transfer into animal T cells and non-replicating cells has proven difficult due to a variety of factors. Currently used methods of retroviral transduction into such cells have a number of practical limitations. Such limitations are compounded by the relatively low titers generally obtained with most retroviral vectors, typically in the range of 10⁵ to 10⁶ infectious virions per milliliter.

The range of host cells that may be infected by a retrovirus or retroviral vector is determined in part by the viral envelope protein. Therefore, a lack or deficiency of the receptor for the given envelope protein would limit transduction efficiency. In addition, a lack of the requisite cellular factors involved in viral binding, penetration, uncoating of the retroviral vectors, viral replication or integration would limit transduction efficiency. In addition, the low titers of available vectors have necessitated methods using co-cultivation with vector producing cells. Alternatively, it has been necessary to add large volumes of vector preparations to the culture medium containing the cells to be transduced to achieve useful transduction frequencies. This leads to a disturbance of the culture conditions for the target cells. These and other problems are addressed by the instant invention.

It is the object of the present invention to provide efficient ex vivo methods for using compositions of high titer recombinant retroviral particles to deliver vector constructs encoding genes of interest to T cells, non-replicating cells, and cells resistant to standard transduction techniques. The transduced cells may then be re-administered to the patient by standard techniques, e.g., intravenous infusion to achieve a desired therapeutic benefit.

### SUMMARY OF THE INVENTION

The present invention provides compositions and methods for transducing T cells, non-dividing cells, or cells resistant to standard transduction techniques comprising obtaining a population of T cells, non-dividing cells, or cells resistant to standard transduction techniques with a preparation of high titer recombinant retroviral particles substantially free from contamination with replication competent retrovirus, wherein the recombinant retroviral particles carry a vector construct encoding a gene of interest.

In another aspect of the invention, an in vivo delivery vehicle comprising transplantable T cells, non-dividing cells, or cells resistant to standard transduction techniques which express a therapeutically effective amount of a gene product encoded by a gene wherein the gene does not occur in T cells, non-dividing cells, or cells resistant to standard transduction techniques or where the gene occurs in T cells, non-dividing cells, or cells resistant to standard transduction techniques but is not expressed in them at levels which are biologically significant or wherein the gene occurs in T cells, non-dividing cells, or cells resistant to standard transduction techniques and has been modified to express in T cells, non-dividing cells, or cells resistant to standard transduction techniques and wherein the gene can be modified to be expressed in T cells, non-dividing cells, or cells resistant to standard transduction techniques is provided.

Within another embodiment of the invention wherein the vector construct encodes a molecule selected from the group consisting of a protein, an active portion of a protein and a RNA molecule with intrinsic biological activity. The protein or active portion of a protein is selected from the group consisting of a cytokine, a colony stimulating factor, a clotting factor, and a hormone.

Within still another embodiment the population of T cells, non-dividing cells, or cells resistant to standard transduction techniques is obtained from an animal. In another embodiment the animal is a human suffering from a disease characterized as a disease selected from the group consisting of a genetic disease, cancer, an infectious disease, a degenerative disease, an inflammatory disease, a cardiovascular disease, and an autoimmune disease.

Within still another embodiment methods are provided for treating diseases such as a genetic disease, cancer, an infectious disease, a degenerative disease, an inflammatory disease, a cardiovascular disease, or an autoimmune disease by administering to a patient a composition or re-introduction of a therapeutically effective amount of the population of transduced T cells, non-dividing cells, or cells resistant to standard transduction techniques. In another another embodiment the T cells, non-dividing cells, or cells resistant to standard transduction techniques are expanded in vitro prior to re-introduction of the cells into the patient.

In other aspects of the invention the transduced T cells, non-dividing cells, or cells resistant to standard transduction techniques and compositions of transduced T cells, non-dividing cells, or cells resistant to standard transduction techniques encoding a gene of interest are provided The envelope protein of the high titer recombinant retroviral particles is an envelope protein selected from the group consisting of a retroviral amphotropic envelope protein, a retroviral ecotropic envelope protein, a retroviral polytropic envelope protein, a retroviral xenotropic envelope protein, a gibbon ape leukemia virus envelope protein and a VSV-g protein. Other retroviral envelope proteins known to those of skill in the art can also be used.

Definition of Terms

The following terms are used throughout the specification. Unless otherwise indicated, these terms are defined as follows:

“Event-specific promoter” refers to transcriptional promoter/enhancer or locus defining elements, or other elements which control gene expression as discussed above, whose transcriptional activity is altered upon response to cellular stimuli. Representative examples of such event-specific promoters include thymidine kinase or thymidylate synthase promoters, a or b interferon promoters and promoters that respond to the presence of hormones (either natal, synthetic or from other non-host organisms).

“Tissue-specific promoter” refers to transcriptional promoter/enhancer or locus defining elements (eg. locus control elements), or other elements which control gene expression as discussed above, which are preferentially active in a limited number of hematopoietic tissue types. Representative examples of such hematopoietic tissue-specific promoters include but not limited to the IgG promoter, α or β globin promoters, and T-cell receptor promoter.

“Transduction” involves the association of a replication defective, recombinant retroviral particle with a cellular receptor, followed by introduction of the nucleic acids carried by the particle into the cell. “Transfection” refers to a method of physical gene transfer wherein no retroviral particle is employed.

“Vector construct”, “retroviral vector”, “recombinant vector”, and “recombinant retroviral vector” refer to a nucleic acid construct capable of directing the expression of a gene of interest. The retroviral vector must include at least one transcriptional promoter/enhancer or locus defining element(s), or other elements which control gene expression by other means such as alternate splicing, nuclear RNA export, post-translational modification of messenger, or post-transcriptional modification of protein. In addition, the retroviral vector must include a nucleic acid molecule which, when transcribed, is operably linked to a gene of interest and acts as a translation initiation sequence. Such vector constructs must also include a packaging signal, long terminal repeats (LTRs) or portion thereof, and positive and negative strand primer binding sites appropriate to the retrovirus used (if these are not already present in the retroviral vector). Optionally, the vector construct may also include a signal which directs polyadenylation, as well as one or more restriction sites and a translation termination sequence. By way example, such vectors will typically include a 5′ LTR, a tRNA binding site, a packaging signal, an origin of second strand DNA synthesis, and a 3′ LTR or a portion thereof. In order to express a desired gene product from such a vector, a gene of interest encoding the desired gene product is also included.

A “RNA molecule with intrinsic biological activity” includes antisense RNA molecules and ribozymes and RNA molecules that bind proteins.

As used herein, “cells resistant to standard transduction techniques” are cells which, in the presence of recombinant retroviral particles according to the invention which have titers of about 10⁶ cfu/ml, as measured on a standard titering cell line such as HT1080, transduce fewer than about 5% ofthe cells. Such cells may include normal cells as well as those which are diseased, such as tumor cells and infected cells, among others.

A “preparation” of high titer recombinant retroviral particles refers to a liquid or lyophilized composition comprising such particles. Preferably, the preparation is equivalent to a pharmaceutical composition in terms of its constituents, but, as those in the art will appreciate, when administration is to cells other than for later human administration, such preparations need not be of pharmaceutical quality, and may in fact comprise only crude, high titer retroviral vector supernatants produced in accordance with the methods described herein.

The term “T cells, non-dividing cells, or cells resistant to standard transduction techniques” includes T cells, B cells, monocytic cells, dendritic cells, nerve stem cells, liver stem cells, intestinal stem cells, bone stem cells, kidney stem cells, skin stem cells, hair stem cells, non-dividing stem cells, non-dividing pancreas cells, non-dividing kidney cells, germ cells and other cells resistant to standard transduction techniques. Progeny cells and precursor cells to the above cell ropes are also encompassed by this term, including T cell precursors and B cell precursors. As used herein, the terms “T cell precursor” and “B cell precursor” refer to all precursor cells that are committed to the T-cell differentation pathway or the B cell differentiation pathway, respectively, but exclude non-commited cells such as hematopoietic stem cells.

Numerous aspects and advantages of the invention will be apparent to those skilled in the art upon consideration of the following detailed description which provides illumination of the practice of the invention.

### DETAILED DESCRIPTION OF THE INVENTION

The present invention is based on the unexpected discovery that recombinant retroviral particles of high titer which carry a vector construct comprising one or more genes of interest can be used ex vivo to efficiently transduce T cells, non-dividing cells, and cells which are resistant to standard transduction techniques. As a result, recombinant retroviral particles according to the invention can be used for purposes of gene therapy and to transduce cells formerly considered to be difficult or impossible to transduce with a retrovirus. A more thorough description of such recombinant retroviral particles, their production and packaging, and uses therefore is provided below.

Generation of Recombinant Retroviral Vectors

As noted above, the present invention provides compositions and methods comprising recombinant retroviral particles for use in ex vivo somatic gene therapy. The construction of recombinant retroviral vectors and particles is described in greater detail in an application entitled “Recombinant Retroviruses” (U.S. Ser. No. 07/586,603, filed Sep. 21, 1990, which is hereby incorporated by reference in its entirety). Production of transduction competent recombinant retroviral particles is described in U.S. Ser. No. 07/800,921 and U.S. Ser. No. 07/800,921, which are hereby incorporated by reference in their entirety. In general, the recombinant vector constructs described herein are prepared by selecting a plasmid with a strong promoter, and appropriate restriction sites for insertion of DNA sequences of interest downstream from the promoter.

According to the invention, the recombinant vector construct is carried by a recombinant retrovirus. Retroviruses are RNA viruses with a single positive strand genome which in general, are nonlytic. Upon infection, the retrovirus reverse transcribes its RNA into DNA, forming a provirus which is inserted into the host cell genome. The retroviral genome can be divided conceptually into two parts. The “trans-acting” portion consists of the region coding for viral structural proteins, including the group specific antigen (gag) gene for synthesis of the core coat proteins; the pol gene for the synthesis of the reverse transcriptase and integrase enzymes; and the envelope (env) gene for the synthesis of envelope glycoproteins. The “cis-acting” portion consists of regions of the genome that is finally packaged into the viral particle. These regions include the packaging signal, long terminal repeats (LTR) with promoters and polyadenylation sites, and two start sites for DNA replication. The internal or “trans-acting” part of the cloned provirus is replaced by the gene of interest to create a “vector construct”. When the vector construct is placed into a cell where viral packaging proteins are present (see U.S. Ser. No. 07/800,921), the transcribed RNA will be packaged as a viral particle which, in turn, will bud off from the cell. These particles are used to transduce tissue cells, allowing the vector construct to integrate into the cell genome. Although the vector construct express its gene product, the virus carrying it is replication defective because the trans-acting portion of the viral genome is absent. Various assays may be utilized in order to detect the presence of any replication competent infectious retrovirus. One preferred assay is the extended S⁺L⁻ assay described below.

In the broadest terms, the retroviral vectors of the invention comprise a transcriptional promoter/enhancer or locus defining element(s), or other elements which control gene expression by other means such as alternate splicing, nuclear RNA export, post-translational modification of messenger, or post-transcriptional modification of protein. In addition, the retroviral vector must include a nucleic acid molecule which, when transcribed in the gene of interest, is operably linked thereto and acts as a translation initiation sequence. Such vector constructs must also include a packaging signal, long terminal repeats (LTRs) or portion thereof, and positive and negative strand primer binding sites appropriate to the retrovirus used (if these are not already present in the retroviral vector). Optionally, the vector construct may also include a signal which directs polyadenylation, as well as one or more restriction sites and a translation termination sequence. By way example, such vectors will typically include a 5′ LTR, a tRNA binding site, a packaging signal, an origin of second strand DNA synthesis, and a 3′ LTR or a portion thereof Such vectors do not contain one or more of a complete gag, pol, or env gene, thereby rendering them replication incompetent. In addition, nucleic acid molecules coding for a selectable marker are neither required nor preferred.

Preferred retroviral vectors contain a portion of the gag coding sequence, preferably that portion which comprises a splice donor and splice acceptor site, the splice acceptor site being positioned such that it is located adjacent to and upstream from the gene of interest. In a particularly preferred embodiment, the gag transcriptional promoter is positioned such that an RNA transcript initiated therefrom contains the 5′ gag UTR and the gene of interest. As an alternative to the gag promoter to control expression of the gene of interest, other suitable promoters, some of which are described below, may be employed. In addition, alternate enhancers may be employed in order to increase the level of expression of the gene of interest.

In preferred embodiments of the invention, retroviral vectors are employed, particularly those based on Moloney murine leukemia virus (MoMLV). MoMLV is a murine retrovirus which has poor infectivity outside of mouse cells. The related amphotropic N2 retrovirus will infect cells from human, mouse and other organisms. Other preferred retroviruses which may be used is the practice of the present invention include Gibbon Ape Leukemia Virus (GALV) (Todaro, et al., Virology, 67:335, 1975; Wilson, et al., J. Vir., 63:2374, 1989), Feline Immunodeficiency Virus TV) (Talbatt, et al., Proc. Nat'l. Acad Sci. USA, 86:5743, 1984), and Feline Leukemia Vies (FeLV) (Leprevette, et al., J. Vir., 50:884, 1984; Elder, et al., J. Vir., 46:871, 1983; Steward, et al., J. Vir., 58:825, 1986; Riedel, et al., J. Vir., 60:242, 1986), although retroviral vectors according to the invention derived from other type C or type D retroviruses or lentiviruses or spuma viruses (see Weiss, RNA Tumor Viruses, vols. I and II, Cold Spring Harbor Laboratory Press, N.Y.) can also be generated.

A varierty of promoters can be used in the vector constructs of the invention, including but not necessarily limited to the cytomegalovirus major immediate early promoter (CMV MIE), the early and late SV40 promoters, the adenovirus major late promoter, thymidine kinase or thymidylate synthase promoters, a or b interferon promoters, event or tissue specific promoters, etc. Promoters may be chosen so as to potently drive high levels of expression or to produce relatively weak expression, as desired. As those in the art will appreciate, numerous RNA polymerase II and RNA polymerase III dependent promoters can be utilized in practicing the invention.

In one embodiment, recombinant retroviral vectors comprising a gene of interest are under the transcriptional control of an event-specific promoter, such that upon activation of the event-specific promoter the gene is expressed. Numerous event-specific promoters may be utilized within the context of the present invention, including for example, promoters which are activated by cellular proliferation (or are otherwise cell-cycle dependent) such as the thymidine kinase or thymidylate synthase promoters (Merrill, Proc. Natl. Acad Sci. USA, 86:4987, 1989; Deng, et al., Mol. Cell. Biol., 9:4079, 1989); or the transferrin receptor promoter, which will be transcriptionally active primarily in rapidly proliferating cells (such as hematopoietic cells) which contain factors capable of activating transcription from these promoters preferentially to express gene products from gene of interest; promoters such as the a or b interferon promoters which are activated when a cell is infected by a virus (Fan and Maniatis, EMBO J., 8:101, 1989; Goodbourn, et al., Cell, 45:601, 1986); and promoters which are activated by the presence of hormones, e.g., estrogen response promoters. See Toohey et al., Mol. Cell. Biol., 6:4526, 1986.

In another embodiment, recombinant retroviral vectors are provided which comprise a gene of interest under the transcriptional control of a tissue-specific promoter, such that upon activation of the tissue-specific promoter the gene is expressed. A wide variety of tissue-specific promoters may be utilized within the context of the present invention. Representative examples of such promoters include: B cell specific promoters such as the IgG promoter; T-cell specific promoters such as the T-cell receptor promoter (Anderson, et al., Proc. Natl. Acad Sci. USA, 85:3551, 1988; Winoto and Baltimore, EMBO J., 8:29, 1989); bone-specific promoters such as the osteocalcin promoter (Markose, et al., Proc. Natl. Acad. Sci. USA, 87:1701, 1990; McDonnell, et al., Mol. Cell. Biol, 9:3517, 1989; Kerner, et al., Proc. Natl. Acad. Sci. USA, 86:4455, 1989), the IL-2 promoter, IL-2 receptor promoter, and the MHC Class II promoter, and hematopoietic tissue specific promoters, for instance erythoid specific-transcription promoters which are active in erythroid cells, such as the porphobilinogen deaminase promoter (Mignotte, et al., Proc. Natl. Acad Sci. USA, 86:6458. 1990), a or b globin specific promoters (van Assendelft. et al., Cell, 56:969, 1989, Forrester, et al., Proc. Natl. Acad. Sci. USA, 86:5439, 1989), endothelial cell specific promoters such as the vWf promoter, magakaryocyte specific promoters such as b-thromboglobulin, and many other tissue-specific promoters.

Retroviral vectors according to the invention may also contain a non-LTR enhancer or promoter, e.g., a CMV or SV40 enhancer operably associated with other elements employed to regulate expression of the gene of interest Additionally, retroviral vectors from which the 3′ LTR enhancer has been deleted, thereby inactivating the 5′ LTR upon integration into a host cell genome, are also contemplated by the invention. A variety of other elements which control gene expression may also be utilized within the context of the present invention including, for example, locus-defining elements including locus control regions, such as those from the b-globin gene and CD2, a T cell marker. In addition, elements which control expression at the level of splicing, nuclear export, and/or translation may also be included in the retroviral vectors. Representative examples include the b-globin intron sequences, the rev and rre elements from HIV-1, the constitutive transport element (CTE) from Mason-Pfizer monkey virus (MPMV), a 219 nucleotide sequence that allows rev-independent replication of rev-negative HIV proviral clones, and a Kodak sequence. Rev protein functions to allow nuclear export of unspliced and singly spliced HIV RNA molecules. The MPMV element allows nuclear export of intron-containing mRNA The CTE element maps to MPMV nucleotides 8022-8240 a (Bray, et al., Biochemistry, 91:1256, 1994).

In another preferred embodiment, the retroviral vector contains a splice donor (SD) site and a splice acceptor (SA) site, wherein the SA is located upstream of the site where the gene of interest is inserted into the recombinant retroviral vector. In a prefered embodiment, the SD and SA sites will be separated by a short, i.e., less than 400 nucleotide, intron sequence. Such sequences may serve to stabilize RNA transcripts. Such stabilizing sequences typically comprise a SD-intron-SA configuration located 5′ to the gene of interest.

The recombinant retroviral vectors of the invention will also preferably contain transcriptional promoters derived from the gag region operably positioned such that a resultant transcript comprising the gene of interest further comprises a 5′ gag UTR (untranslated region) upstream of the gene of interest.

The present invention also provides for multivalent vector constructs, the construction of which may require two promoters when two proteins are being expressed, because one promoter may not ensure adequate levels of gene expression of the second gene. In particular, where the vector construct expresses an antisense message or ribozyme, a second promoter may not be necessary. Within certain embodiments, an internal ribosome binding site (IRBS) or herpes simplex virus thymidine kinase (HSVTK) promoter is placed in conjunction with the second gene of interest in order to boost the levels of gene expression of the second gene. Briefly, with respect to IRBS, the upstream untranslated region of the immunoglobulin heavy chain binding protein has been shown to support the internal engagement of a bicistronic message (Jacejak, et al., Nature 353:90, 1991). This sequence is small, approximately 300 base pairs, and may readily be incorporated into a vector in order to express multiple genes from a multi-cistronic message whose cistrons begin with this sequence.

Retroviral vector constructs according to the invention will often be encoded on a plasmid, a nucleic acid molecule capable of propogation, segregation, and extrachromosomal maintenance upon introduction into a host cell. As those in the art will understand, any of a wide range of existing or new plasmids can be used in the practice of the invention. Such plasmids contain an origin of replication and typically are modified to contain a one or more multiple cloning sites to facilitate recombinant use. Preferably, plasmids used in accordance with the present invention will be capable of propogation in both eukaryotic and prokaryoric host cells.

Generation of Packaging Cells

Another aspect of the invention relates to methods of producing recombinant xenotropic retroviral particles incorporating the retroviral vectors described herein. In one embodiment, vectors are packaged into infectious virions through the use of a packaging cell. Briefly, a packaging cell is a cell comprising, in addition to its natural genetic complement, additional nucleic acids coding for those retroviral structural polypeptides required to package a retroviral genome, be it recombinant (i.e., a retroviral vector) or otherwise. The retroviral particles are made in packaging cells by combining the retroviral genome with a capsid and envelope to make a transduction competent, preferably replication defective, virion. Briefly, these and other packaging cells will contain one, and preferably two or more nucleic acid molecules coding for the various polypeptides, e.g., gag, pol, and env, required to package a retroviral vector into an infectious virion. Upon introduction of a nucleic acid molecule coding for the retroviral vector, the packaging cells will produce infectious retroviral particles. Packaging cell lines transfected with a retroviral vector according to the invention which produce infectious virions are referred to as “producer” cell lines.

A wide variety of animal cells may be utilized to prepare the packaging cells of the present invention, including without limitation, epithelial cells, fibroblasts, hepatocytes, endothelial cells, myobiasts, astrocytes, lymphocytes, etc.. Preferentially, cell lines are selected that lack genomic sequences which are homologous to the retroviral vector construct, gag/pol expression cassette and env expression cassette to be utilized. Methods for determining homology may be readily accomplished by, for example, hybridization analysis (Martin et al., Proc. Natl. Acad. Sci., USA, vol. 78:4892-96, 1981; and U.S. Ser. No. 07/800,921, supra).

The most common packaging cell lines (PCLs) used for MoMLV vector systems (psi2, PA12, PA317) are derived from murine cell lines. However, murine cell lines are typically not the preferred choice to produce retroviral vectors intended for human therapeutic use because such cell lines are known to: contain endogenous retroviruses, some of which are closely related in sequence and retroviral type to the MLV vector system preferred for use in practicing the present invention; contain non-retroviral or defective retroviral sequences that are known to package efficiently; and cause deleterious effects due to the presence of murine cell membrane components.

An important consideration in developing packaging cell lines useful in the invention is the production therefrom of replication incompetent virions, or avoidance of generating replication-competent retrovirus (RCR) (Munchau et al., Virology. vol. 176:262-65, 1991). This will ensure that infectious retroviral particles harboring the recombinant retroviral vectors of the invention will be incapable of independent replication in target cells, be they in vitro or in vivo. Independent replication, should it occur, may lead to the production of wild-type virus, which in turn could lead to multiple integrations into the chromosome(s) of a patient's cells, thereby increasing the possibility of insertion al mutagenesis and its associated problems. RCR production can occur in at least two ways: (1) through homologous recombination between the therapeutic proviral DNA and the DNA encoding the retroviral structural genes (“gag/pol” and “env”) present in the packaging cell line; and (2) generation of replication-competent virus by homologous recombination of the proviral DNA with the very large number of defective endogenous proviruses found in murine packaging cell lines.

To circumvent inherent safety problems associated with the use of murine based recombinant retroviruses, as are preferred in the practice of this invention, packaging cell lines may be derived from various non-murine cell lines. These include cell lines from various mammals, including humans, dogs, monkeys, mink, hamsters, and rats. As those in the art will appreciate, a multitude of packaging cell lines can be generated using techniques known in the art (for instance, see U.S. Ser. No. 08/156,789 and U.S. Ser. No. 08/136,739). In preferred embodiments, cell lines are derived from canine or human cell lines, which are known to lack genomic sequences homologous to tat of MoMLV by hybridization analysis (Martin et al., supra). A particularly preferred parent dog cell line is D17 (A.T.C.C. accession no. CRL 8543). HT-1080 (A.T.C.C. accession no. CCL 121; Graham et al., Vir., vol. 52:456, 1973) and 293 cells (Felgner et al., Proc. Nat'l. Acad. Sci. USA 84:7413, 1987) represent particularly preferred parental human cell lines. Construction of packaging cell lines from these cell lines for use in conjunction with a MoMLV based recombiant retroviral vector is described in detail in U.S. Ser. No. 08/156,789, supra.

Thus, a desirable prerequisite for the use of retroviruses in gene therapy is the availability of retroviral packaging cell lines incapable of producing replication competent, or “wild-type,” virus. As packaging cell lines contain one or more nucleic acid molecules coding for the structural proteins required to assemble the retroviral vector into infectious retroviral particles, recombination events between these various constructs might produce replication competent virus, i.e., infectious retroviral particles containing a genome encoding all of the structural genes and regulatory elements, including a packaging signal, required for independent replication. In the past several years, many different constructions have been developed in an attempt to obviate this concern. Such constructions include: deletions in the 3′ LTR and portions of the 5′ LTR (see. Miller and Buttimore, Mol. Cell. Biol., vol. 6:2895-2902, 1986), where two recombination events are necessary to form RCR, use of complementay portions of helper virus, divided among two separate plasmids, one containing gag and pol, and the other containing env (see, Markowitz et al., J. Virol., vol. 62:1120-1124; and Markowitz et al., Virology, vol 167:600-606, 1988), where three recombination events are required to generate RCR.

The ability to express gag/pol and env function separately allows for manipulation of these functions independently. A cell line that expresses ample amounts of gag/pol can be used, for example, to address questions of titre with regard to env. One factor resulting in measured low titres is the density of appropriate receptor molecules on the target cell or tissue. A second factor is the affinity of the receptor for the retroviral xenotropic envelope protein. One report suggests that xenotropic vector, in the presence of replication-complement xenotropic virus, may more effectively infect human hematopoietic progenitor cells (Eglitis, et al., Biochem. Biophys. Res. Comm. 151:201-206, 1988). Xenotropic vector-containing particles, in the presence of replication-competent xenotropic virus, also infect cells from other species which are not easily injectable by amphotropic virus such as bovine, porcine, and equine cells (Delouis, et al., Biochem. Biophys Res. Comm. 169:80-14, 1990). In a preferred embodiment of the invention, packaging cell lines which express a xenotropic env gene are provided. Significantly, recombinant retroviral particles produced from such packaging cell lines are substantially free from association with replication competent retrovirus (“RCR”).

More recently, further improved methods and compositions for inhibiting the production of replication incompetent retrovirus have been developed. See co-owned U.S. Ser. No. 09/028,126, filed Sep. 7, 1994. Briefly, the spread of replication competent retrovirus generated through recombination events between the recombinant retroviral vector and one or more of the nucleic acid constructs coding for the retroviral structural proteins may be prevented by providing vectors which encode a non-biologically active inhibitory molecule, but which produce a nucleic acid molecule encoding a biologically active inhibitory molecule in the event of such recombination. The expression of the inhibitory molecule prevents production of RCR either by killing the producer cell(s) in which that event occurred or by suppressing production of the retroviral vectors therein. A variety of inhibitory molecules may be used, including ribozymes, which cleave the RNA transcript of the replication competent virus, or a toxin such as ricin A, tetanus, or diphtheria toxin, herpes thymidine kinase, etc. As those in the art will appreciate, the teachings there a may be readily adapted to the present invention.

In addition to issues of safety, the choice of host cell line for the packaging cell line is of importance because many of the biological properties (such as titer) and physical properties (such as stability) of retroviral particles are dictated by the properties of the host cell. For instance, the host cell must efficiently express (transcribe) the vector RNA genome, prime the vector for first strand synthesis with a cellular tRNA, tolerate and covalently modify the MLV structural proteins (proteolysis, glycosylation, myristylation, and phosphorylation), and enable virion budding from the cell membrane. For example, it has been found that vector made from the mouse packaging line PA317 is retained by a 0.3 micron filter, while that made from a CA line will pass through Furthermore, sera from primates, including humans, but not that from a wide variety of lower mammals or birds, is known to inactivate retroviruses by an antibody independent complement lysis method. Such activity is non-selective for a variety of distantly related retroviruses. Retroviruses of avian, murine (including MoMLV), feline, and simian origin are inactivated and lysed by normal human serum. See Welsh et al., (1975) Nature, vol. 257:612-614; Welsh et al., (1976) Virology, vol. 74:432-440; Banapour et al., (1986) Virology, vol. 152:268-271; and Cooper et al., (1986) Immunology of the Complement System, Pub. American Press, Inc., pp:139-162. In addition, replication competent murine amphotropic retroviruses injected intravenously into primates in vivo are cleared within 15 minutes by a process mediated in whole or in part by primate complement (Cornetta et al. (1990), Human Gene Therapy, vol. 1:15-30; Cornetta et al. (1991), Human Gene Therapy, vol. 2:5-14). However, it has recently been discovered that retroviral resistance to complement inactivation by human serum is mediated, at least in some instances, by the packaging cell line from which the retroviral particles were produced. Retroviruses produced from various human packaging cell lines were resistant to inactivation by a component of human serum, presumably complement, but were sensitive to serum from baboons and macques. See commonly owned U.S. Ser. No. 08/367,071, filed on Dec. 30, 1994. Thus, in a preferred embodiment of the invention, recombinant retroviral particles coding for full length factor VIII are produced in human packaging cell lines, with packaging cell lines derived from HT1080 or 293 cells being particularly preferred.

In addition to generating infectious, replication defective recombinant retroviruses as described above, at least two other alternative systems can be used to produce recombinant retroviruses carrying the vector construct. One such system (Webb, et al., BBRC, 190:536, 1993) employs the insect virus, baculovirus, while the other takes advantage of the mammalian viruses vaccinia and adenovirus (Pavirani, et al., BBRC, 145:234, 1987). Each of these systems can make large amounts of any given protein for which the gene has been cloned. For example, see Smith, et al. (Mol. Cell. Biol., 3:12, 1983); Piccini. et al. (Meth. Enzymology. 153:545, 1987); and Mansour et. al. (Proc. Natl. Acad. Sci. USA, 82:1359, 1985). These viral vectors can be used to produce proteins in tissue culture cells by insertion of appropriate genes and, hence, could be adapted to make retroviral vector particles from tissue culture. In an adenovirus system, genes can be inserted into vectors and used to express proteins in mammalian cells either by in vitro construction (Ballay, et al., 4:3861, 1985) or by recombination in cells (Thummel, et al., J. Mol. Appl. Genetics, 1:435, 1982).

An alternative approach involves cell-free packaging systems. For instance, retroviral structural proteins can be made in a baculovirus system (or other protein production systems, such as yeast or E. coli) in a similar manner as described in Smith et al. (supra). Recombinant retroviral genomes are made by in vitro RNA synthesis (see, for example, Flamant and Sorge, J. Virol., 62:1827, 1988). The structural proteins and RNA genomes are then mixed with tRNA, followed by the addition of liposomes with embedded env protein and cell extracts (typically from mouse cells) or purified components (which provide env and other necessary processing, and any or other necessary cell-derived functions). The mixture is then treated (e.g. by sonication, temperature manipulation, or rotary dialysis) to allow encapsidation of nascent retroviral particles. This procedure allows production of high titer, replication incompetent recombinant retroviruses without contamination with pathogenic retroviruses or replication-competent retroviruses.

Another important factor to consider in the selection of a packaging cell line is the viral titer produced therefrom following introduction of a nucleic acid molecule from which the retroviral vector is produced. Many factors can limit viral titer. One of the most significant limiting factors is the expression level of the packaging proteins gag, pol, and env. In the case of retroviral particles, expression of retroviral vector RNA from the provirus can also significantly limit titer. In order to select packaging cells and the resultant producer cells expressing high levels of the required products, an appropriate titering assay is required. As described in greater detail below, a suitable PCR-based titering assay can be utilized.

In addition to preparing packaging and producer cell lines which supply proteins for packaging that are homologous for the backbone of the viral vector, e.g., retroviral gag, pol, and env proteins for packaging of a retroviral vector, packaging and producer systems which result in chimeric viral particles, for instance a MoMLV-based retroviral vector packaged in a DNA virus capsid, may also be employed. Many other packaging and producer systems based on viruses unrelated to that of the viral vector can also be utilized, as those in the art will appreciate.

Altering the Host Range of Recombinant Retroviral Particles

Another aspect of the invention concerns recombinant xenotropic retroviral particles which have an altered host range as compared to retroviral particles containing amphotropic envelope proteins. The host cell range specificity of a retrovirus is determined in part by the env gene products present in the lipid envelope. Interestingly, envelope proteins from one retrovirus can often substitute, to varying degrees, for that of another retrovirus, thereby altering host range of the resultant vector. Thus, packaging cell lines (PCLs) have been generated to express either amphotropic, ecotropic, xenotropic, polytropic, or other envelope tropisms. Additionally, retroviruses according to the invention which contain “hybrid” or “chimeric” xenotropic envelope proteins can be similarly generated. Retroviral particles produced from any of these packaging cell lines can be used to infect any cell which contains the corresponding distinct receptor (Rein and Schultz, Virology, 136:144, 1984).

The assembly of retroviruses is characterized by selective inclusion of the retroviral genome and accessory proteins into a budding retroviral particle. Interestingly, envelope proteins from non-murine retrovirus sources can be used for pseudotyping (i.e., the encapsidation of viral RNA from one species by viral proteins of another species) a vector to alter its host range. Because a piece of cell membrane buds off to form the retroviral envelope, molecules normally in the membrane may be carried along on the viral envelope. Thus, a number of different potential ligands can be put on the surface of retroviral particles by manipulating the packaging cell line in which the vectors are produced or by choosing various types of cell lines with particular surface markers.

Briefly, in this aspect the present invention provides for enveloped retroviral particles comprising: a nuleocapsid including nuleocapsid protein having an origin from a first virus, which is a retrovirus; a packageable nucleic acid molecule encoding a gene of interest associated with the nuleocapsid; and a membrane-associated xenotropic protein which determines a host range.

In another preferred form of the present invention, the membrane-associated envelope protein of the vector particles is a chimeric or hybrid protein including an exterior receptor binding domain and a membrane-associated domain from a xenotropic envelope protein, at least a portion of the exterior receptor binding domain being derived from a different origin than at least a portion of the membrane-associated domain. The chimeric protein is preferably derived from two origins, wherein no more than one of the two origins is retroviral.

Another embodiment of this aspect of the present invention concerns cell lines that produce the foregoing vector particles. Preferably, such cell lines are stably transfected with a nucleic acid molecule encoding the membrane-associated protein, whose expression is driven by an inducible promoter.

Retroviral particles according to the invention may be targeted to a specific cell type by including in the retroviral particles a component, most frequently a polypeptide or carbohydrate, which binds to a cell surface receptor specific for that cell type. Such targeting may be accomplished by preparing a packaging cell line which expresses a chimeric env protein comprising a portion of the env protein required for vial particle assembly in conjunction with a cell-specific binding domain. In another embodiment, env proteins from more than one viral type may be employed, such that resultant viral particles contain more than one species of env proteins. Yet another embodiment involves inclusion of a cell specific ligand in the retroviral capsid or envelope to provide target specificity. In a preferred embodiment at this aspect of the invention, the env gene employed encodes all or a portion of the env protein required for retroviral assembly in conjunction with a receptor binding domain of a polypeptide ligand known to interact with a cell surface receptor whose tissue distribution is limited to the cell type(s) to be targeted, e.g., a T cell. In this regard, it may be preferable to utilize a receptor binding domain which binds receptors expressed at high levels on the target cell's surface.

In order to control the specific site of integration into a patient's genome in those instances where the vector construct employed leads to integration of the viral genome into a chromosome of the recipient cell, as occurs in the case of retroviral infection, homologous recombination or use of a modified integrase enzyme which directs insertion to a specific site can be utilized. Approaches for the use of integrase proteins to direct site specific integration is described in WO 91/02805 entitled “Recombinant Retroviruses Delivering Vector Constructs to Target Cells” and co-owned U.S. application Ser. No. 445,466, filed May 22, 1995, both of which are hereby incorporated by reference. Such site-specific insertion of the vector carrying the gene of interest may provide for gene replacement therapy, reduced chances of insertion al mutagenesis, minimize interference from other sequences present in the patient's DNA, and allow insertion at specific target sites to reduce or eliminate expression of an undesirable gene (such as a viral or tumorigenic gene) in the patients DNA.

Non-viral membrane-associated proteins may also be used to enhance targeting of recombinant retroviral particles, including xenotrophic retroviral particles, to T cells. Representative examples include polypeptides which act as ligands for T cell surface receptors. Depending on the tissue distribution of the receptor for the protein in question, the recombinant xenotropic retroviral particle could be targeted to a different subset of T cells.

When a ligand to be included within the envelope is not a naturally occurring membrane-associated proteins it is necessary to associate the ligand with the membrane, preferably by making a “hybrid” or “chimeric” envelope protein. It is important to understand that such hybrid envelope proteins can contain extracellular domains from proteins other than other viral or retroviral env proteins. To accomplish this, the gene coding for the ligand can be functionally combined with sequences coding for a membrane-associated domain of the env protein. By “naturally occurring membrane associated protein”, it is meant those proteins that in their native state exist in vivo in association with lipid membrane such as that found associated with a cell membrane or on a viral envelope. As such, hybrid envelopes can be used to tailor the tropism (and effectively increase titers) of a retroviral vector according to the invention, as the extracellular component of env proteins is responsible for specific receptor binding. The cytoplasmic domain of these proteins, on the other hand, play a role in virion formation. The present invention recognizes that numerous hybrid env gene products (i.e., specifically, retroviral env proteins having cytoplasmic regions and extracellular binding regions which do not naturally occur together) can be generated and may alter host range specificity.

In a preferred embodiment, this is accomplished by recombining the gene coding for the ligand (or part thereof conferring receptor binding activity) proximate of the membrane-binding domain of the envelope proteins that stably assemble with a given capsid protein. The resulting construct will code for a bifunctional chimeric protein capable of enhanced cell targeting and inclusion in a retroviral lipid envelope.

Vector particles having non-native membrane-associated ligands as described herein, will, advantageously, have a host range determined by the ligand-receptor interaction of the membrane-associated protein. Thus, for targeted delivery to T cells, a vector particle having altered host range can be produced using the methods of the present invention. The ligand will be selected to provide a host range including T cells. Many different targeting strategies can be employed in connection with this aspect of the invention.

Antibodies may be also utilized to target a selected cell type, such as anti-CD4 antibodies to target CD4+ T-cells and anti-CD8 antibodies to target CD8+ cells (see generally, Wilchek, et al., Anal. Biochem.. 171:1, 1988).

T lymphocytes or T cells are non-antibody producing lymphocytes that constitute the part of the cell-mediated arm of the immune system. T cells arise from immature lymphocytes that migrate from the bone marrow to the thymus, where they undergo a maturation process under the direction of thymic hormones. Here, the immature lymphocytes rapidly divide increasing to enormous numbers. The maturing T cells become immunocompetent by having the ability to recognize and bind a specific antigen. Activation of immunocompetent T Cells is triggered by antigen binding to the lymphocyte's surface receptors.

T cells can be isolated by a variety of procedures known to those skilled in the art. For example, crude T cell suspensions can be prepared from spleen and lymph nodes by passing homogenates through nylon wool columns (Current Protocols in Immunology, Coligan, et. al. (1992) Green Publishing Associates and Wiley-Interscience, New York). This procedure offers a convenient means of enriching T cell populations through the removal of accessory and B cells. T cells from mouse spleen and lymph node do not express the cell-surface glycoproteins encoded for by MHC class II genes, whereas most non-T cells do. Therefore, T cell enrichment can be accomplished by the elimination of non-T cells using anti-MHC class II monoclonal antibodies. Similarly, other antibodies could be used to deplete specific populations of non-T cells. For example, a-Ig for B cells and a-MacI for macrophages.

T cells can be further fractionated into a number of different subpopulations by techniques known to those skilled in the art. Two major subpopulations can be isolated based on their differential expression of the cell surface markers CD4 and CD8. For example, following the enrichment of T cells as described above, CD4⁺ cells can be enriched through the use of antibodies specific for CD8 (described in Current Protocols in Immunology, supra ). Alternatively, CD4⁺ cells can be enriched through the use of antibodies specific to CD4, coupled to a solid support such as magnetic beads. Conversely, CD8+ cells can be enriched through the use of antibodies specific for CD4, or can be isolated by the use of CD8 antibodies coupled to a solid support. CD4 lymphocytes from HIV-1 infected patients can be expanded ex vivo, before or after transduction, as described by Wilson et. al. (J. Infect Dis 172:88, 1995).

Following purification of T cells, a variety of methods of transduction known to those skilled in the art can be performed. For example, one such approach involves transduction of the purified T cell population with vector containing supernatant cultures derived from vector producing cells. A second approach involves co-cultivation of an irradiated monolayer of vector producing cells with the purified T cells. A third approach involves a similar co-cultivation approach, however the purified T cells are pre-stimulated with various cytokines and cultured 48 hours prior to the co-cultivation with the irradiated vector producing cells. Pre-stimulation prior to transduction increases effective gene transfer (Nolta et al., Exp. Hematol. 20:1065; 1992). While not wishing to be bound by theory, the increased level of transduction is attributed to increased proliferation of the T cells necessary for efficient retroviral transduction. Stimulation of these cultures to proliferate also provides increased cell populations for re-infusion into the patient.

Subsequent to co-cultivation, T cells are collected from the vector producing cell monolayer, expanded, and frozen in liquid nitrogen. The expression of vector in tranduced cells can be assessed by a number of assays known to those skilled in the art. For example, Western blot or Northern analysis can be employed depending on the nature of the inserted gene of interest. Once expression has been established and the transformed T cells have been tested for the presence of adventitious agents, they are infused back into the patient via the peripheral blood stream.

Those in the art will also recognize that it is also possible to add ligand molecules exogenously to the retroviral particles which are either incorporated into the lipid envelope or which can be linked chemically to the lipid or protein constituents thereof. In addition, a wide variety of high affinity binding pairs can be used as targeting elements. Representative examples of include biotin/avidin with an affinity (K_{D}) of 10⁻¹⁵ M (Richards, Meth. Enz., 184:3, 1990; Green, Adv. in Protein Chem., 29:85, 1985) and cystatin/papain with an affinity of 10⁻¹⁴ M (Bjork, et al., Biochemistry, 29:1770, 1990). A wide variety of other high affinity binding pairs may also be developed, for example, by preparing and selecting antibodies which recognize a selected T cell antigen with high affinity (see generally, U.S. Pat. Nos. RE 32,011, 4,902,614, 4,543,439, and 4,411,993; see also Monoclonal Antibodies, Hybridomas: A New Dimension in Biological Analyses, Plenum Press, Kennett, McKearn, and Bechtol, eds, 1980, and Antibodies: A Laboratory Manual, Harlow and Lane eds., Cold Spring Harbor Laboratory Press, 1988). The binding pair for such antibodies, typically other antibodies or antibody fragments, may be produced by recombinant techniques (see Huse, et al., Science, 246:1275, 1989; see also Sastry, et al., Proc. Natl. Acad. Sci. USA, 86:5728, 1989; and Michelle Alting-Mees, et al., Strategies in Molecular Biology, 3:1, 1990).

As will be evident to one of ordinary skill in the art given the disclosure provided herein, either member (or molecule) of the affinity binding pair may be coupled to the retroviral particle. Nevertheless, within preferred embodiments of the invention, the larger of the two affinity binding pairs (e.g., avidin of the avidin/biotin pair) is coupled to the retroviral particle. As utilized within the context of targeting, the term “coupled” may refer to either noncovalent or covalent interactions, although generally covalent bonds are preferred. Numerous coupling methods may be utilized, including, for example, use of crosslinking agents such as N-succinimidyl-3-(2-pyridyl dithio) propionate (“SPDP”; Carlson, et al., J. Biochem., 173:723, 1978) and other such compounds known in the art.

In particularly preferred embodiments of the invention, a member of the high affinity binding pair is either expressed on, or included as an integral part of, a retroviral particle, e.g., in the retroviral lipid envelope. For example, a member of the high affinity binding pair may be co-expressed with the envelope protein as a hybrid protein or expressed from an appropriate vector which targets the member of the high affinity binding pair to the cell membrane in the proper orientation.

Uses of Recombinant Retroviral Particles

In one aspect, the present invention provides methods for inhibiting the growth of a selected tumor (“cancer”) in an human, comprising the step of transducing T cells ex vivo with a vector construct which directs the expression of at least one anti-tumor agent. Within the context of the present invention, “inibiting the growth of a selected tumor” refers to either (1) the direct inhibition of tumor cell division, or (2) immune cell mediated tumor cell lysis, or both, which leads to a suppression in the net expansion of tumor cells. Inhibition of tumor growth by either of these two mechanisms may be readily determined by one of ordinary skill in the art based upon a number of well known methods, for example, by measuring the tumor size over time, such as by radiologic imaging methods (e.g., single photon and positron emission computerized tomography; see generally, “Nuclear Medicine in Clinical Oncology,” Winkler, C. (ed) Springer-Verlag, New York, 1986) or by a variety of imaging agents, including, for example, conventional imaging agents (e.g., Gallium-67 citrate) or specialized reagents for metabolite imaging, receptor imaging, or immunologic imaging. In addition, non-radioactive methods such as ultrasound (see, “Ultrasonic Differential Diagnosis of Tumors”, Kossoff and Fukuda, (eds.), Igaku-Shoin, New York, 1984), may also be utilized to estimate tumor size. Alternatively, for other forms of cancer, inhibition of tumor growth may be determined based upon a change in the presence of a tumor marker, e.g., prostate specific antigen (“PSA”) for the detection of prostate cancer (see U.S. Pat. No. Re. 33,405), and Carcino-Embryonic Antigen (“CEA”) for the detection of colorectal and certain breast cancers. For yet other types of cancers such as leukemia, inhibition of tumor growth may be determined based upon decreased numbers of leukemic cells in a representative blood cell count.

Within the context of the present invention, “anti-tumor agent” refers to a compound or molecule which inhibits tumor growth. Representative examples of anti-tumor agents include immune activators and tumor proliferation inhibitors. Briefly, immune activators function by improving immune recognition of tumor-specific antigens such that the immune system becomes “primed.” Priming may consist of lymphocyte proliferation, differentiation, or evolution to higher affinity interactions. The immune system thus primed will more effectively inhibit or kill tumor cells. Immune activation may be subcategorized into immune modulators (molecules which affect the interaction between lymphocyte and tumor cell) and lymphokines, that act to proliferate, activate, or differentiate immune effector cells. Representative examples of immune modulators include CD-3, ICAM-1, ICAM-2, LFA-1, LFA-3, b-2-microglobulin, chaperones, alpha interferon, gamma interferon, B7/BB1 and major histocompatibility complex (MHC), and T cell receptor proteins or synthetic equivalents such as T cell receptors with modified recognition sites. Representative examples of lymphokines include gamma interferon, tumor necrosis factor, IL-1, IL-2, IL-3, IL4, IL-5, IL6, L-7, IL-8, IL-9, IL-10, IL-11, GM-CSF, CSF-1, and G-CSF. In addition, RNA molecules having intrinsic biological activity may be utilized as anti-tumor agents.

Sequences which encode anti-tumor agents may be obtained from a variety of sources. For example, plasmids that contain sequences which encode anti-tumor agents may be obtained from a depository such as the American Type Culture Collection (ATCC, Rockville, Md.), or from commercial sources such as British Bio-Technology Limited (Cowley, Oxford England). Alternatively, known cDNA sequences which encode anti-tumor agents may be obtained from cells which express or contain the sequences. Additionally, cDNA or mRNA libraries from specific cell sources can be purchased from commercial sources from which the desired sequences can be readily cloned by conventional techniques, e.g., PCR amplification. Sequences which encode anti-tumor agents may also be synthesized, for example, on an Applied Biosystems Inc. DNA synthesizer (e.g., ABI DNA synthesizer model 392, Foster City, Calif.).

In addition to the anti-tumor agents described above, the present invention also provides anti-tumor agents which comprise a fusion protein of, for example, two or more cytokines, immune modulators, toxins or differentiation factors. Preferred anti-tumor agents in this regard include alpha interferon—Interleukin-2, GM-CSF—IL4, GM-CSF—IL-2, GM-CSF—IL-3 (see U.S. Pat. Nos. 5,082,927 and 5,108,910), GM-CSF—gamma interferon, and gamma interferon—IL4, with gamma interferon—Interleukin-2 being particularly preferred.

Within another embodiments, the anti-tumor agent may further comprise a membrane anchor. The membrane anchor may be selected from a variety of sequences, including, for example, the transmembrane domain of well known proteins. Generally, membrane anchor sequences are regions of a protein that anchor the protein to a membrane. Customarily, there are two types of anchor sequences that attach a protein to the outer surface of a cell membrane: (1) transmembrane regions that span the lipid bilayer of the cell membrane (proteins containing such regions are referred to integral membrane proteins); and (2) domains which interact with an integral membrane protein or with the polar surface of the membrane (such proteins are referred to as peripheral, or extrinsic, proteins).

Membrane anchors derived from integral membrane proteins are preferred. Membrane spanning regions typically have a similar structure, with a 20 to 25 amino-acid residue portion consisting almost entirely of hydrophobic residues located inside the membrane (see Eisenberg et al., Ann. Rev. Biochem. 53:595-623, 1984). Membrane spanning regions typically have an alpha helical structure (see Eisenberg et al. supra; Heijne and Manoil at supra). Within a preferred embodiment, a membrane anchor is fused to the C-terminus of gamma interferon fusion protein, wherein the membrane anchor comprises the gamma-chain of the Fc receptor.

Tumorigenicity of an anti-tumor agent can be assessed by various assays. Representative assays include tumor formation in nude mice or rats, colony formation in soft agar, and preparation of transgenic animals, such as transgenic mice. In addition to tumorgenicity studies, it is generally preferable to determine the toxicity of an anti-tumor agent. A variety of methods well known to those of skill in the art may be utilized to measure such toxicity, including for example, clinical chemistry assays which measure the systemic levels of various proteins and enzymes, as well as blood cell volume and number. Once an anti-tumor agent has been selected, it is placed into a vector construct according to the invention.

Such a vector construct can then be packaged into a recombinant retroviral vector and be used to transduce ex vivo T cells which are then re-introduced into the patient. In the context of the present invention, it should be understood that the removed cells may not only be returned to the same patient, but may also be utilized to inhibit the growth of selected tumor cells in another allogeneic human.

Within one embodiment, the recombinant vector construct directs the expression of a protein or active portion of a protein that binds to newly synthesized MHC class I molecules intracellularly. This binding prevents migration of the MHC class I molecule from the endoplasmic reticulum, resulting in the inhibition of terminal glycosylation. This blocks transport of these molecules to the cell surface and prevents cell recognition and lysis by CTL. For instance, one of the products of the E3 gene may be used to inhibit transport of MHC class I molecules to the surface of the transformed cell. More specifically, E3 encodes a 19 kD transmembrane glycoprotein, E3/19K, transcribed from the E3 region of the adenovirus 2 genome. Within the context of the present invention, a multivalent recombinant viral vector construct is administered directly or indirectly, and contains a gene encoding a therapeutic protein and the E3/19K sequence, which upon expression, produces the therapeutic protein and the E3/19K protein. The E3/19K protein inhibits the surface expression of MHC class I surface molecules, including those MHC molecules that have bound peptides of the therapeutic protein. Consequently, cells transformed by the vector evade an immune response against the therapeutic protein they produce.

Within another embodiment of the present invention, the multivalent recombinant vector construct directs the expression of a therapeutic protein and a protein or an active portion of a protein capable of binding β₂-microglobulin. Transport of MHC class I molecules to the cell surface for antigen presentation requires association with β₂-microglobulin. Thus, proteins that bind β₂-microglobulin and inhibit its association with MHC class I indirectly inhibit MHC class I antigen presentation. Suitable proteins include the H301 gene product. Briefly, the H301 gene, obtained from the human cytomegalovirus (CMV) encodes a glycoprotein with sequence homology to the β₂-microglobulin binding site on the heavy chain of the MHC class I molecule (Browne et al., Nature 347:770, 1990). H301 binds β₂-microglobulin, thereby preventing the maturation of MHC class I molecules, and renders transformed cells unrecognizable by cytotoxic T-cells, thus evading MHC class I restricted immune surveillance.

Other proteins, rot discussed above, that function to inhibit or down-regulate MHC class I antigen presentation either generally or more specifically for the specific foreign protein encoded may also be identified and utilized within the context of the present invention. In order to identify such proteins, in particular those derived from mammalian pathogens (and, in turn, active portions thereof such as the EBNA-1 gly-ala repeat from EBV virus), a recombinant vector construct that expresses a protein or an active portion thereof either as a separate entity or fused to the active protein suspected of being capable of inhibiting MHC class I antigen presentation is transformed into a tester cell line, such as the murine cell line BC10ME (see WO 91/02805, entitled “Recombinant Retroviruses Delivering Vector Constructs to Target Cells”). The tester cell lines with and without the sequence encoding the candidate protein are compared to stimulators and/or targets in the CTL assay. A decrease in cell lysis correponding to the transformed tester cell indicates that the candidate protein is capable of inhibiting MHC presentation.

An alternative method to determine down-regulation of MHC class I surface expression is by FACS analysis. More specifically, cell lines are transformed with a recombinant vector construct encoding the candidate protein. After drug selection and expansion, the cells are analyzed by FACS for MHC class I expression and compared to that of non-transformed cells. A decrease in cell surface expression of MHC class I indicates that the candidate protein is capable of inhibiting MHC presentation.

Any of the gene delivery vehicles described above may include, contain (and/or express) one or more heterologous sequences. A wide variety of heterologous sequences may be utilized within the context of the present invention, including for example, cytotoxic genes, disease-associated antigens, antisense sequences, sequences which encode gene products that activate a compound with little or no cytotoxicity (i.e., a “prodrug”) into a toxic product, sequences which encode immiunogenic portions of disease-associated antigens and sequences which encode immune accessory molecules. Representative examples of cytotoxic genes include the genes which encode proteins such as ricin (Lamb et al., Eur. J. Biochem. 148:265-270, 1985), abrin (Wood et al., Eur. J. Biochem. 198:723-732, 1991; Evensen, et al., J. of Biol. Chem. 266:6848-6852, 1991: Collins et al., J. of Biol. Chem. 265:8665-8669, 1990; Chen et al., Fed. of Eur. Biochem Soc. 309:115-118, 1992), diphtheria toxin (Tweten et al., J. Biol. Chem. 260:10392-10394, 1985), cholera toxin (Mekalanos et al., Nature 306:551-557, 1983; Sanchez & Holmgren, PNAS 86:481-485, 1989), gelonin (Stirpe et al., J. Biol. Chem. 255:6947-6953, 1980), pokeweed (Irvin. Pharmac. Ther. 21:371-387, 1983), antiviral protein (Barbieri et al., Biochem. J. 203:55-59, 1982; Irvin et al., Arch. Biochem. & Biophys. 200:418-425, 1980; Irvin, Arch. Biochem. & Biophys. 169:522-528, 1975), tritin, Shigella toxin (Calderwood et al., PNAS 84:4364-4368, 1987; Jackson et al., Microb. Path. 2:147-153, 1987), and Pseudomonas exotoxin A (Carroll and Collier, J. Biol. Chem. 262:8707-8711, 1987).

Within further embodiments of the invention, antisense RNA may be utilized as a cytotoxic gene in order to induce a potent Class I restricted response. Briefly, in addition to binding RNA and thereby preventing translation of a specific mRNA, high levels of specific antisense sequences may be utilized to induce the increased expression of interferons (including γ-interferon), due to the formation of large quantities of double-stranded RNA. The increased expression of gamma interferon (γ-IFN), in turn, boosts the expression of MHC Class I antigens. Preferred antisense sequences for use in this regard include actin RNA, myosin RNA, and histone RNA. Antisense RNA which forms a mismatch with actin RNA is particularly preferred.

Within other embodiments of the invention, antisense sequences are provided which inhibit, for example, tumor cell growth, viral replication, or a genetic disease by preventing the cellular synthesis of critical proteins needed for cell growth. Examples of such antisense sequences include antisense thymidine kinase, antisense dihydrofolate reductase (Maher and Dolnick. Arch. Biochem. & Biophys. 253:214-220, 1987; Bzik et al., PNAS 84:8360-8364, 1987), antisense HER2 (Coussens et al., Science 230:1132-1139, 1985), antisense ABL (Faistein, et al., Oncogene 4:1477-1481, 1989), antisense Myc (Stanton et al., Nature 310:423-425, 1984) and antisense ras, as well as antisense sequences which block any of the enzymes in the nucleotide biosynthetic pathway.

Within other aspects of the invention, gene delivery vehicles are provided which direct the expression of a gene product that activates a compound with little or no cytotoxicity (i.e., a “prodrug”) into a toxic product. Representative examples of such gene products include varicella zoster virus thymidine kinase (VZVTK), herpes simplex virus thymidine kinase HSVTK) (Field et al., J. Gen. Virol. 49:115-124, 1980), and E. coli. guanine phosphoribosyl transferase (see U.S. patent application Ser. No. 08/155,944, entitled “Compositions and Methods for Utilizing Conditionally Lethal Genes,” filed Nov. 18, 1993; see also WO 93/10218 entitled “Vectors Including Foreign Genes and Negative Selection Markers”, WO 93/01281 entitled 37 Cytosine Deaminase Negative Selection System for Gene Transfer Techniques and Therapies”, WO 93/08843 entitled “Trapped Cells and Use Thereof as a Drug”, WO 93/08844 entitled “Transformant Cells for the Prophylaxis or Treatment of Diseases Caused by Viruses, Particularly Pathogenic Retroviruses”, and WO 90/07936 entitled “Recombinant Therapies for Infection and Hyperproliferative Disorders.”) Within preferred embodiments of the invention, the gene delivery vehicle directs the expression of a gene product that activates a compound with little or no cytotoxicity into a toxic product in the presence of a pathogenic agent, thereby affecting localized therapy to the pathogenic agent (see U.S. Ser. No. 08/155,944).

Within one embodiment of the invention, gene delivery vehicles are provided which direct the expression of a HSVTK gene downstream, and under the transcriptional control of an HIV promoter (which is known to be transcriptionally silent except when activated by HIV tat protein). Briefly, expression of the tat gene product in human cells infected with HIV and carrying the gene delivery vehicle causes increased production of HSVTK. The cells (either in vitro or in vivo) are then exposed to a drug such as ganciclovir, acyclovir or its analogues (FIAU, FIAC, DHPG). Such drugs are known to be phosphorylated by HSVTK (but not by cellular thymidine kinase) to their corresponding active nucleotide triphosphate forms. Acyclovir and FIAU triphosphates inhibit cellular polymerases in general, leading to the specific destruction of cells expressing HSVTK in transgenic mice (see Borrelli et al., Proc. Natl. Acad Sci. USA 85:7572, 1988). Those cells containing the gene delivery vehicle and expressing HIV tat protein are selectively killed by the presence of a specific dose of these drugs.

Within further aspects of the present invention, gene delivery vehicles of the present invention may also direct the expression of one or more sequences which encode immunogenic portions of disease-associated antigens. As utilized within the context of the present invention, antigens are deemed to be “disease-associated” if they are either associated with rendering a cell (or organism) diseased or are associated with the disease-state in general but are not required or essential for rendering the cell diseased. In addition, antigens are considered to be “immunogenic” if they are capable, under appropriate conditions, of causing an immune response (either cell-mediated or humoral). Immunogenic “portions” may be of variable size, but are preferably at least 9 amino acids long, and may include the entire antigen.

A wide variety of “disease-associated” antigens are contemplated within the scope of the present invention, including for example immunogenic, non-tumorigenic forms of altered cellular components which are normally associated with tumor cells (see U.S. Ser. No. 08/104,424). Representative examples of altered cellular components which are normally associated with tumor cells include ras* (wherein “*” is understood to refer to antigens which have been altered to be non-tumorigenic), p53*, Rb*, altered protein encoded by Wilms' tumor gene, ubiquitin*, mucin, protein encoded by the DCC. APC, and MCC genes, as well as receptors or receptor-like structures such as neu, thyroid hormone receptor, platelet derived growth factor (“PDGF”) receptor, insulin receptor, epidermal growth factor (“EGF”) receptor, and the colony stimulating factor (“CSF”) receptor.

“Disease-associated” antigens should also be understood to include all or portions of various eukaryotic (including for example, parasites), prokaryotic (e.g., bacterial) or viral pathogens. Representative examples of viral pathogens include the hepatitis B virus (“HBV”) and hepatitis C virus (“HCV”; see U.S. Ser. No. 08/102/132), human papiloma virus (“HPV”; see WO 92/05248; WO 90/0459; EPO 133,123), Epstein-Barr virus (“EBV”; see EPO 173,254; JP 1,128,788; and U.S. Pat. Nos. 4,939,088 and 5,173,414), feline leukemia virus (“FeLV”; see U.S. Ser. No. 07/948,358; EPO 377,842; WO 90/08832; WO 93/09238), feline immunodeficiency virus (“FIV”; U.S. Pat. No. 5,037,753; WO 92/15684; WO 90/13573; and JP 4,126,085), HTLV I and II, and human immunodeficiency virus (“HIV”; see U.S. Ser. No. 07/965,084).

Within other aspects of the present invention, the gene delivery vehicles described above may also direct the expression of one or more immune accessory molecules. As utilized herein, the phrase “immune accessory molecules” refers to molecules which can either increase or decrease the recognition, presentation or activation of an immune response (either cell-mediated or humoral). Representative examples of immune accessory molecules include IL-1, IL-2, IL-3, IL4, IL-5, IL6, IL-7 (U.S. Pat. No. 4,965,195), IL-8, IL-9, IL-10, IL-11, IL-12. IL-13, IL-14, and IL-15. (Wolf et al.., J. Immun. 46:3074, 1991; Gubler et al., PNAS 88:4143, 1991; WO 90/05147; EPO 433,827), IL-13 (WO 94/04680), GM-CSF, M-CSF-1, G-CSF, CD3 (Krissanen et al., Immunogenetics 26:258-266, 1987), CD8, ICAM-1 (Simmons et al., Nature 331:624-627, 1988), ICAM-2 (Singer, Science 255: 1671, 1992), b2-microglobulin (Parnes et al., PNAS 78:2253-2257, 1981), LFA-1 (Altmann et al, Nature 338: 521, 1989), LFA-3 (Wallner et al., J. Exp. Med 166(4)-923-932, 1987), HLA Class I, HLA Class II molecules B7 (Freeman et al., J. Immun. 143:2714, 1989), and B7-2. Within a preferred embodiment, the heterologous gene encodes g-IFN.

Within preferred aspects of the present invention, the gene delivery vehicles described herein may direct the expression of more than one heterologous sequence. Such multiple sequences may be controlled either by a single promoter, or preferably, by additional secondary promoters (e.g., internal ribosome binding sites or “IRBS”). Within preferred embodiments of the invention, a gene delivery vehicle directs the expression of heterologous sequences which act synergistically. For example, within one embodiment retrovector constructs are provided which direct the expression of a molecule such as IL-12, IL-2, γ-IFN, or other molecule which acts to increase cell-mediated presentation in the T_{H}1 pathway, along with an immunogenic portion of a disease-associated antigen. In such embodiments, immune presentation and processing of the disease-associated antigen will be increased due to the presence of the immune accessory molecule.

Within other aspects of the invention, gene delivery vehicles are provided which direct the expression of one or more heterologous sequences which encode “replacement” genes. As utilized herein, it should be understood that the term “replacement genes” refers to a nucleic acid molecule which encodes a therapeutic protein that is capable of preventing, inhibiting, stabilizing or reversing an inherited or noninherited genetic defect. Representative examples of such genetic defects include disorders in metabolism, immune regulation, hormonal regulation, and enzymatic or membrane associated structural function. Representative examples of diseases caused by such defects include cystic fibrosis (due to a defect in the cystic fibrosis transmembrane conductance regulator (“CFTCR”), see Dorin et al, Nature 326:614, ), Parkinson's Disease, adenosine deaminase deficiency (“ADA”; Hahma et al., J. Bact. 173:3663-3672, 1991), β-globin disorders, hemophilia A & B (Factor VIII-deficiencies; see Wood et al., Nature 312:330, 1984), Gaucher disease, diabetes, forms of gouty arthritis and Lesch-Nylan disease (due to “HPRT” deficiencies: see Jolly et al., PNAS 80:477-481, 1983) Duchennes muscular dystrophy and familial hypercholesterolemia (LDL Receptor mutations; see Yamamoto et al., Cell 39:27-38, 1984).

As is described herein, T cell populations transduced ex vivo with retroviral vectors expressing a variety of different proteins can be re-introduced into a patient in order to treat a variety of different disorders. For instance, HIV and other viral infections of T cells can be treated by this method can be used in the treatment of viral infections of T cells, including HIV infections. In particular with regard to HIV infection, a number of differenct theraputic approaches can be used. For example, T cells can be tranduced ex vivo with a high titer preparation of a retroviral vector expressing a nucleic acid or protein which interferes with HBV replication (Baltimore, D. Nature 335:395, 1988). In particular, retroviral vectors expressing mutant HIV nucleic acid sequences, ribozymes, antisense molecules, and proteins which can interfere with HIV infection and replication can be produced as described in WO 91/02805, entitled “Recombinant Retroviruses Delivering Vector Constructs to Target Cells”, and in WO 92/05266, entitled “Packaging Cells”, both of which publications are hereby incorporated by reference.

T cell populations obtained from patients with a variety of disorders can be transduced ex vivo with high titer preparations of retroviral vectors expressing a protein which is effective for treatment of the disorder when present in the bloodstream. The recombinant vector construct can express at least one therapeutic protein selected from the group consisting of factor VIII, factor IX, hemoglobin, phenylalanine hydroxylase, adenosine deaminase, hypoxanthine-guanine phosphoribosyltransferase, a₁-antitrypsin, transmembrane conductance regulator, and glucocerebrosidase. The transduced T cells can then be reintroduced into the patient where they secrete the beneficial protein into the blood of the patient or the activity of the protein detoxifies an agent responsible for the disease (eg. adenosine in ADA deficiency or glucocereberoside in Gaucher's syndrome). This approach can be used, for example in the treatment of a variety of genetic disorders, included those listed above. For instance, T cells can be obtained from a hemophilia patient and tranduced ex vivo with a retroviral vector expressing factor VII. A number of different factor VIII nucleic acid acid constructs can be used. For example, retroviral vectors expressing full-length factor VIII or a functional factor VIII protein lacking the B domain can be produced as described in Example 2 herein. In addition, a variety of different retroviral vectors constructs expressing full-length factor VIII proteins can be produced as described in co-pending U.S. application Ser. No. 08/366,851, which is hereby incorporated by reference.

As described herein, T cells, non-dividing cells, and other cells traditionally resistant to transduction with retroviral vectors can be successfully transduced ex vivo with high titer preparations of retroviral vectors. In particular, retroviral vectors expressing a protein converting a prodrug to a toxic molecule can be used alone or in addition to a therapeutic protein. As described above, the re-introduction of cells transduced with such vectors are usefull in the treatment of a variety of disorders. In addition, this approach can be used to modulate the activity of transduced T cells or other transduced cells when they are introduced into a patient, by the introduction of the prodrug in vivo.

The term “modulate the activity” as used herein, includes the inhibition of a cellular function by prodrug administration. This modulation of activity can be accomplished, for example, by the killing of the tranduced cells by the activated prodrug. For example, allogeneic bone marrow transplants are used in treatment of cancers such as leukemias. In addition, T cells from the donor are infused in order to aid engraftment and to increase the anti-tumor immune response. However, a proportion of patients treated with this allogeneic transplantation can develop graft vs. host disease. Ex vivo transduction of the T cells with retroviral vectors transduced with a retroviral vector encoding a protein capable of activating a prodrug provides a mechanism to modulate the activity of the T cells after they are introduced into the patient. In particular, the resultant graft versus host disease can be reduced or eliminated by administration of the prodrug to the patient. A variety of different proteins, such as herpes thymidine kinase, which are capable of converting a prodrug to a toxic molecule can be used. Representative examples of such gene products include varicella zoster virus thymidine kinase (VZVTK), herpes simplex virus thymidine kinase (HSVTK) (Field el al., J. Gen. Virol. 49:115-124, 1980), and E. coli. guanine phosphoribosyl tansferase (see U.S. patent application Ser. No. 08/155,944, entitled “Compositions and Methods for Utilizing Conditionally Lethal Genes,” filed Nov. 18, 1993 and incorporated herein by reference; see also WO 93/10218 entitled “Vectors Including Foreign Genes and Negative Selection Markers”, WO 93/01281 entitled “Cytosine Deaminase Negative Selection System for Gene Transfer Techniques and Therapies”, WO 93/08843 entitled “Trapped Cells and Use Thereof as a Drug”, WO 93/08844 entitled “Transformant Cells for the Prophylaxis or Treatment of Diseases Caused by Viruses, Particularly Pathogenic Retroviruses”, and WO 90/07936 entitled “Recombinant Therapies for Infection and Hyperproliferative Disorders.”)

Sequences which encode the above-described heterologous genes may be readily obtained from a variety of sources. For example, plasmids which contain sequences that encode immune accessory molecules may be obtained from a depository such as the American Type Culture Collection (ATCC, Rockville, Md.), or from commercial sources such as British Bio-Technology Limited (Cowley, Oxford, England). Representative sources sequences which encode the above-noted immune accessory molecules include BBG 12 (containing the GM-CSF gene coding for the mature protein of 127 amino acids), BBG 6 (which contains sequences encoding γ-IFN), ATCC No. 39656 (which contains sequences encoding TNF), ATCC No. 20663 (which contains sequences encoding a-IFN), ATCC Nos. 31902, 31902 and 39517 (which contains sequences encoding b-IFN), ATCC No 67024 (which contains a sequence which encodes IL-1), ATCC Nos. 39405, 39452, 39516, 39626 and 39673 (which contains sequences encoding IL-2), ATCC Nos. 59399, 59398. and 67326 (which contain sequences encoding IL-3), ATCC No. 57592 (which contains sequences encoding IL-4), ATCC Nos. 59394 and 59395 (which contain sequences encoding IL-5), and ATCC No. 67153 (which contains sequences encoding IL-6). It will be evident to one of skill in the art that one may utilize either the entire sequence of the protein, or an appropriate portion thereof which encodes the biologically active portion of the protein.

Alternatively, know cDNA sequences which encode heterologous genes may be obtained from cells which express or contain such sequences. Briefly, within one embodiment mRNA from a cell which expresses the gene of interest is reverse transcribed with reverse transcriptase using oligo dT or random primers. The single stranded cDNA may then be amplified by PCR (see U.S. Pat. Nos. 4,683,202, 4,683,195 and 4,800,159. See also PCR Technology: Principles and Applications for DNA Amplification. Erlich (ed.), Stockton Press. 1989 all of which are incorporated by DNA Amplification, Erlich (ed), Stockton Press, 1989 all of which are incorporated by reference herein in their entirety) utilizing oligonucleotide primers complementary to sequences on either side of desired sequences. In particular, a double stranded DNA is denatured by heating in the presence of heat stable Taq polymerase, sequence specific DNA primers, ATP, CTP, GTP and TTP. Double-standed DNA is produced when synthesis is complete. This cycle may be repeated many times, resulting in a factorial amplification of the desired DNA.

Sequences which encode the above-described genes may also be synthesized, for example, on an Applied Biosystems Inc. DNA synthesizer (e.g., ABI DNA synthesizer model 392 (Foster City, Calif.)).

Preparation and Purification of Recombinant Retroviral Particles

Another aspect of the invention concerns the preparation of recombinant retroviral particles. Recombinant retroviral particles according to the invention can be produced in a variety of ways, as those in the art will appreciate. For example, producer cells, i.e., cells containing all necessary components for retroviral vector packaging (including a nucleic acid molecule encoding the retroviral vector), can be grown in roller bottles, in bioreactors, in hollow fiber apparatus, and in cell hotels. Cells can be maintained either on a solid support in liquid medium, or grown as suspensions. A wide variety of bioreactor configurations and sizes can be used in the practice of the present invention.

Cell factories (also termed “cell hotels”) typically contain 2, 10, or 40 trays, are molded from virgin polystyrene, treated to provide a Nuclon D surface, and assembled by sonic welding one to another. Generally, these factories have two port tubes which allow access to the chambers for adding reagents or removing culture fluid. A 10-layer factory provides 6000 cm² of surface area for growing cells, roughly the equivalent of 27 T-225 flasks. Cell factories are available from a variety of manufacturers, including for example Nunc. Most cell types are capable of producing high titer vector for 3-6 days, allowing for multiple harvests. Each cell type is tested to determine the optimal harvest time after seeding and the optimal number of harvest days. Cells are typically initially grown in DMEM supplemented with 2-20% FBS in roller bottles until the required number of cells for seeding a cell factory is obtained. Cells are then seeded into the factories and 2 liters of culture supernatant containing vector is harvested later at an appropriate time. Fresh media is used to replenish the cultures.

Hollow fiber culture methods may also be used. Briefly, high titer retroviral production using hollow fiber cultures is based on increasing viral concentration as the cells are being cultured to a high density in a reduced volume of media Cells are fed nutrients and waste products are diluted using a larger volume of fresh media which circulates through the lumen of numerous capillary fibers. The cells are cultured on the exterior spaces of the capillary fibers in a bioreactor chamber where cell waste products are exchanged for nutrients by diffusion through 30 kD pores in the capillary fibers. Retroviruses which are produced from the cell lines are too large to pass through the pores, and thus concentrate in the hollow fiber bioreactor along side of the cells. The volume of media being cultured on the cell side is approximately 10 to 100 fold lower then volumes required for equivalent cell densities cultured in tissue culture dishes or flasks. This decrease fold in volume inversely correlates with the fold induction of titer when hollow fiber retroviral titers are compared to tissue culture dishes or flasks. This 10-100 fold induction in titer is seen when an individual retroviral producer cell line is amenable to hollow fiber growth conditions. To achieve maximum cell density, the individual cells must be able to grow in very close proximity and on top of each other. Many cell lines will not grow in this fashion and retroviral packaging cell lines based on these types of cell lines may not achieve 10 fold increases in titer. Cell lines which would grow very well would be non-adherent cell line and it is believed that a retroviral producer line based on a non-adherent cell line may reach 100 fold increases in titer compared to tissue culture dishes and flasks.

Regardless of the retroviral particle and production method, high titer (from about 10⁷-10¹¹ cfu/mL) stocks can be prepared that will cause high level expression of the desired products upon introduction into appropriate cells. When all components required for retroviral particle assembly are present, high-level expression will occur, thereby producing high titer stocks. And while high titer stocks are preferred, retroviral preparations having titers ranging from about 10³ to 10⁶ cfu/mL may also be employed, although retroviral titers can be increased by various purification methods, as described below.

After production by an appropriate means, the infectious recombinant xenotropic retroviral particles may be preserved in a crude or purified form. Crude retroviral particles are produced by cultivated infected cells, wherein retroviral particles are released from the cells into the culture media. The virus may be preserved in crude form by first adding a sufficient amount of a formulation buffer to the culture media containing the recombinant virus to form an aqueous suspension.

Recombinant retroviral particles can also be preserved in a purified form. More specifically, prior to the addition of formulation buffer, the crude retroviral preparation described above is clarified by passing it through a filter, and then concentrated, such as by a cross flow concentrating system (Filtron Technology Corp., Nortborough, Mass.). Within one embodiment, DNase is added to the concentrate to digest exogenous DNA. The digest is then diafiltrated a to remove excess media components and establish the recombinant virus in a more desirable buffered solution. The diafiltrate is then passed over a gel filtration column, such as a Sephadex S-500 gel column, and the purified recombinant virus is eluted.

Crude recombinant xenotropic retroviral preparations can also be purified by ion exchange column chromatography, such as is described in more detail in U.S. Ser. No. 08/093,436. In general, the crude preparation is clarified by passing it through a filter, and the filtrate loaded onto a column containing a highly sulfonated cellulose matrix, wherein the amount of sulfate per gram of cellulose ranges from about 6-15 μg. The recombinant retrovirus is eluted from the column in purified form by using a high salt buffer. The high salt buffer is then exchanged for a more desirable buffer by passing the eluate over a molecular exclusion column. The purified preparation may then be formulated or stored, preferably at −70° C.

Additionally, the preparations containing recombinant retroviruses according to the invention can be concentrated during purification in order to increase the titer of recombinant retrovirus. A wide variety of methods may be utilized for increasing retroviral concentration, including for example, precipitation of recombinant retroviruses with ammonium sulfate, polyethylene glycol (“PEG”) concentration, concentration by centrifugation (either with or without gradients such as PERCOLL, or “cushions” such as sucrose, use of concentration filters (e.g. Amicon filtration), and 2-phase separations.

Briefly, to accomplish concentration by precipitation of recombinant retroviruses with ammonium sulfate, ammonium sulfate is added slowly to an appropriate concentration, followed by centrifugation and removal of the ammonium sulfate either by dialysis or by separation on a hydrophobic column.

Alternatively, recombinant retroviruses may be concentrated from culture medium with PEG (Green, et al, PNAS 67:385-393, 1970; Syrewicz, et al., Appl. Micro. 24:488-494, 1972). Such methods are rapid, simple, and inexpensive. However, like ammonium sulfate precipitation, use of PEG also concentrates other proteins from solution.

Within other embodiments, recombinant retroviruses may be concentrated by centrifugation, and more particularly, low speed centrifugation, which avoids difficulties associated with pelleting that accompanies high speed centrifugation (e.g., virus destruction or inactivation).

Recombinant retroviruses according to the invention may also be concentrated by an aqueous two-phase separation method. Briefly, polymeric aqueous two-phase systems may be prepared by dissolving two different non-compatible polymers in water. Many pairs of water-soluble polymers may be utilized in the construction of such two-phase systems, including for example polyethylene glycol (“PEG”) or methylcellulose, and dextran or dextran sulfate (see Walter and Johansson, Anal. Biochem. 155:215-242, 1986; Albertsson, “Partition of Cell Particles and Macromolecules” Wiley, New York, 1960). As described in more detail below in Example 7, utilizing PEG at concentrations ranging from 5% to 8% (preferably 6.5%), and dextran sulfate at concentrations ranging from 0.4% to 1% (preferably 0.4%), an aqueous two-phase system may be established suitable for purifing recombinant retroviruses. Utilizg such procedures, approxirate 100-fold concentration can be achieved with yields of approximately 50% or more of the total starting retrovirus.

For purposes of illustration, a representative concentration process which combines several concentration steps is set forth below. Briefly, recombinant retroviruses may be prepared either from roller bottles, cell factories, or bioreactors prior to concentration. Removed media containing the recombinant retrovirus may be frozen at −70° C., or more preferably, stored at 2° C. to 8° C. in large pooled batches prior to processing.

For material obtained from a bioreactor, the recombinant retrovirus pool is first clarified through a 0.8 μm filter (1.2 μm glass fiber pre-filter, 0.8 μm cellulose acetate) connected in series with a 0.65 μm filter. This filter arrangement provides approximately 2 square feet of filter, and allows processing of about 15-20 liters of pooled material before clogging. For material obtained from roller bottles or cell factories, a single 0.65 μm cartridge (2 sq. ft.) normally suffices for volumes up to 40 liters. For 80 liter cell factory processes, a 5 sq. ft. filter may be required.

Preferably, after clarification the filter is rinsed with buffer (e.g., 150 mM NaCl, 25 mM Tris, pH 7.2-7.5). Following clarification, recombinant retroviruses are concentrated by tangential flow ultrafiltration utilizing cassettes with a 300,000 mw cut off. For bioreactor material (containing 12% to 16% FBS), 4-5 L of material may be concentrated per cassette. For roller bottles or cell factories at 12-16% FBS, 5-6 L of material may be concentrated per cassette. Finally, for cell factories containing 10% FBS, 8-9 L of material may be concentrated per cassette. Utilizing such procedures at an appropriate pressure differential between filtrate and retentate, up to 80 liters of material may be concentrated to a volume of less than 500 mL in under two hours. This process also provides a yield of about 80%.

Following the ultrafiltration step, DNAse may be added to a concentration of 50 U/mL, and recirculated at a lower pump speed with the filtrate line closed for 30 minutes. Discontinuous diafiltration is then accomplished by adding additional buffer and utilizing the same cross differential pressure as before. Generally, recovery after this step is approximately 70%.

Concentrated material is then subjected to column chromatography on a Phamacia S-500 HG size exclusion gel, utilizing 50 mM NaCl and 25 mM Tris pH 7.2-7.5 as minimum salt and ionic strength concentrations. Generally, recombinant xenotropic retroviruses elute off in the first peak.

Tangential flow filtration may once again be utilized to further reduce the volume of the preparation, after which the concentrated material is sterilized by filtration through a 0.2 μm Millipore filter.

As an alternative to in vivo production, the retroviral packaging proteins may be produced, together or separately, from appropriate cells. However, instead of introducing a nucleic acid molecule enabling production of the viral vector, an in vitro packaging reaction is conducted comprising the gag, pol, and env proteins, the retroviral vector, tRNA, and other necessary components. The resulting retroviral particles can then purified and, if desired, concentrated.

Formulation of Pharmaceutical Compositions

Another aspect of the invention relates to pharmaceutical compositions comprising recombinant retroviral vectors as described above, in combination with a pharmaceutically acceptable carrier or diluent, while another aspect is directed toward a method for preserving an infectious recombinant retroviruses for subsequent reconstitution such that the recombinant retrovirus is capable of infecting mammalian cells upon reconstitution. The methods described can be used to preserve a variety of different viruses, including recombinant type C retroviruses such as gibbon ape leukemia virus, feline leukemia virus and xeno-, poly- and amphotropic murine leukemia virus (Weiss, et al., RNA Tumor Viruses, 2d ed. 1985). See U.S. Ser. No. 08/153,342.

Pharmaceutically acceptable carriers or diluents are nontoxic to recipients at the dosages and concentrations employed. Representative examples of carriers or diluents for injectable solutions include water, isotonic saline solutions, preferably buffered at a physiological pH (such as phosphate-buffered saline or Tris-buffered saline), mannitol, dextrose, glycerol, and ethanol, as well as polypeptides or proteins such as human serum albumin (HSA). A particularly preferred composition comprises a recombinant xenotropic retrovirus in 10 mg/mL mannitol, 1 mg/mL HSA, 20 mM Tris, pH 7.2. and 150 mM NaCl. In this case, since the recombinant xenotropic retroviral particle represents approximately 1 μg of material, it may be less than 1% of high molecular weight material, and less than 1/100,000 of the total material (including water). This composition is stable at −70° C. for at least six months.

Pharmaceutical compositions of the present invention may also additionally include factors which stimulate T cell division, and hence, uptake and incorporation of vector constructs according to the invention.

Particularly preferred methods and compositions for preserving recombinant retroviruses are described in U.S. Ser. No. 08/135,938, filed Oct. 12, 1993, and U.S. Ser. No. 8/153,342, filed Nov. 15, 1993.

The use of recombinant retroviruses to transduce T cells useful in treating patients requires that the product be able to be transported and stored for long periods at a desired temperature such that infectivity and viability of the recombinant retrovirus is retained. The difficulty of preserving recombinant retroviruses absent low temperature storage and transport presents problems in Third World countries, where adequate refrigeration capabilities are often lacking.

The initial stabilization of materials in dry form to the preservation of antitoxins, antigens and bacteria has been described (Flosodort, et al., J. Immunol., 29:389, 1935). However, a limitation in this process included partial denaturation of proteins when dried from an aqueous state at ambient temperatures. Drying from the frozen state helped reduce this denaturation and led to efficient preservation of other biological materials, including bacteria and viruses (Stamp, et al., J. Gen. Microbiol., 1:251, 1947; Rowe, et al., Virology, 42:136, 1970; and Rowe, et al., Cryobiology, 8:153, 1971). More recently, sugars such as sucrose, raffinose, glucose and trehalose were added in various combinations as stabilizing agents prior to lyophilization of viruses. The use of sugars enhanced recovery of viable viruses, for research purposes which require that only some virus survive for later propagation.

Recombinant retroviruses according to the invention can be stored in liquid, or preferably, lyophilized form. Factors influencing stability include the formulation (liquid, freeze dried, constituents thereof, etc.) and storage conditions, including temperature, storage container, exposure to light, etc. Alternatively, retroviral particles according to the invention can be stored as liquids at low temperatures. In a preferred embodiment, the recombinant retroviruses of the invention are formulated to preserve infectivity in a lyophilized form at elevated temperatures, and for this form to be suitable for injection into patients following reconstitution.

Recombinant retroviral particles comprising retroviral vector constructs according to the invention can be formulated in crude or, preferably, purified form. Crude retroviral preparations may be produced by various cell culture methods, where retroviral particles are released from the cells into the culture media. Recombinant retroviral particles may be preserved in crude form by adding a sufficient amount of formulation buffer. Typically, the formulation buffer is an aqueous solution containing various components, such as one or more saccharides, high molecular weight structural additives, buffering components, and/or amino acids.

The recombinant retroviruses described herein can also be preserved in a purified form. For instance, prior to the addition of formulation buffer, crude preparations as described above may be clarified by filtration, and then concentrated, such as by a cross flow concentrating system (Filtron Technology Corp., Nortborough, Mass.). DNase may be added to the concentrate to digest exogenous DNA, followed by diafiltration to remove excess media components and substitute in a more desirable buffered solution. The diafiltrate may then passed over a gel filtration column, such as a Sephadex® S-500 gel column, and the eluted xenotropic retroviral particles retained. A sufficient amount of formulation buffer may then be added to the eluate to reach a desired final concentration of the constituents and to minimally dilute the retroviral preparation. The aqueous suspension can then be stored, preferably at −70° C., or immediately formulated.

In an alternative procedure, the crude preparation can be purified by ion exchange column chromatography. Briefly, the crude recombinant retrovirus is clarified by filtration and then loaded onto a column comprising a highly sulfonated cellulose matrix. Highly purified recombinant xenotropic retrovirus is eluted from the column using a high salt buffer, which is then exchanged for a more desirable buffer by passing the eluate over a molecular exclusion column. After recover, formulation buffer may then added to adjust the final concentration, as discussed above, followed by low temperature storage, preferably at −70° C. or immediate formulation.

When a dried formulation is desired, an aqueous preparation containing a crude or purified retroviral preparation can be prepared by lyophilization or evaporation. Lyophilization involves cooling the aqueous preparation below the glass transition temperature or below the eutectic point temperature of the solution, and removing water by sublimation. For example, a multistep freeze drying procedure as described by Phillips et al. (Cryobiology, vol. 18:414, 1981) can be used to lyophilize the formulated recombinant virus, preferably from a temperature of −40° C. to −45° C. The resulting composition should contain less than 10% water by weight. Once lyophilized, such a preparation is stable and may be stored at −20° C. to 25° C.

In an evaporative method, water is removed by evaporation from the retroviral preparation aqueous suspension at ambient temperature. Evaporation can be accomplished by various techniques, including spray drying (see EP 520,748), where the preparation is delivered into a flow of preheated gas, usually air, whereupon water rapidly evaporates from droplets of the suspension. Spray drying apparatus are available from a number of manufacturers (e.g., Drytec, Ltd., Tonbridge, England; Lab-Plant, Ltd., Huddersfield, England). Once dehydrated, the recombinant retroviral prearation is stable and may be stored at −20° C. to 25° C. The resulting moisture content of the dried or lyophilized preparation may be determined through use of a Karl-Fischer apparatus (EM Science Aquastar' VIB volumetric titrator, Cherry Hill, N.J.), or through a gravimetric method. Once dehydrated, the recombinant xenotropic retrovirus is stable and may be stored at −20° C. to 25° C.

As mentioned previously, aqueous preparations comprising xenotropic retroviruses according to the invention used for formulation are typically composed of one or more saccharides, high molecular weight structural additives, buffering components, and water, and may also include one or more amino acids. It has been found that the combination of these components acts to preserve the activity of the recombinant retrovirus upon freezing and lyophilization, or drying through evaporation. See co-owned U.S. Ser. No. 08/153,342, filed Nov. 15, 1993. Various saccharides may be used alone or in combination, including sucrose, mannitol, glucose, trehalose, inositol, fructose, maltose, and galactose, with lactose being particularly preferred. The concentration of the saccharide can range from 0.1% to 30% by weight, preferably from about 1% to 12% by weight. A particularly preferred concentration of lactose is 3%-4% by weight. Additionally, saccharide combinations can also be employed, including lactose and mannitol or sucrose and mannitol. It will also be evident to those skilled in the art that it may be preferable to use certain saccharides in the aqueous solution when the lyophilized formulation is intended for room temperature storage. Specifically, disaccharides, such as lactose or trehalose, are preferred for such formulations.

One or more high molecular weight structural additives may be used to aid in preventing retroviral aggregation during freezing and provides structural support in the lyophilized or dried state. In the context of the present invention, structural additives are considered to be of “high molecular weight” if they are greater than 5000 daltons. A preferred high molecular weight structural additive is human serum albumin (HSA), although other substances may also be used, such as hydroxyethyl-cellulose, hydroxymethyl-cellulose, dextran, cellulose, gelatin, povidone, etc. Preferably, the concentration of the high molecular weight structural additive can range from 0.05% to 20%, with 0.1% to 10% by weight being preferred, and a concentration of 0.1% by weight HSA being particularly preferred.

Amino acids, if present, tend to firther preserve retroviral infectivity. In addition, amino acids function to further preserve retroviral infectivity during sublimation of the cooled aqueous suspension and while in the lyophilized state. A preferred amino acid is arginine, but other amino acids such as lysine, ornithine, serine, glycine, glutamine, asparagine, glutamic acid or aspartic acid can also be used. Preferably, the amino acid concentration ranges from 0.1% to 10% by weight. A particularly preferred arginine concentration is 0.1% by weight.

A variety of buffering components may be used to rnaintain a relatively constant pH, depending on the pH range desired, preferably between 7.0 and 7.8. Suitable buffers include phosphate buffer and citrate buffer. A particularly preferred formulation pH is 7.4, and a preferred buffer is tromethamine.

It may also be preferable to include in the formulation a neutral salt to adjust the final is-osmotic salt concentration. Suitable neutral salts include sodium chloride, potassium chloride, and magnesium chloride, with sodium chloride being preferred.

A particularly preferred method of preserving recombinant retroviruses in a lyophilized state for subsequent reconstitution comprises: (a) preparing an aqueous recombinant xetroviral preparation comprising, in addition to the recombinant xenotropic retrovirus, about (i) 4% by weight of lactose, (ii) 0.1% by weight of human serum albumin, (iii) 0.03% or less by weight of NaCl, (iv) 0.1% by weight of arginine, and a sufficient amount of tromethamine to provide a pH of approximately 7.4; (b) cooling the preparation to a temperature of about −40° C. to −45° C. to form a frozen preparation; and (c) removing water from the frozen preparation by sublimation to form a lyophilized composition having less than 2% water by weight. It is preferred that the recombinant xenotropic retrovirus be replication defective and suitable for administration into humans cells upon reconstitution.

The lyophilized or dehydrated retroviruses of the subject invention may be reconstituted using a variety of substances, but are preferably reconstituted using water. In certain instances, dilute salt solutions which bring the final formulation to isotonicity may also be used. In addition, it may be advantageous to use aqueous solutions containing components known to enhance the activity of the reconstituted virus. Such components include cytokines, such as IL-2, polycations, such as protamine sulfate, or other components which enhance the transduction efficiency of the reconstituted virus. Lyophilized or dehydrated recombinant virus may be reconstituted with any convenient volume of water or the reconstituting agents noted above that allow substantial, and preferably total solubilization of the lyophilized or dehydrated sample.

Administration of Recombinant Retroviral Particles

In another aspect of the present invention, methods are provided for treating human patients afflicted with a varierty of diseases, including a genetic disease, cancer, an infectious disease, an autoimmune disease, and inflammatory disease, a cardiovascular disease, and a degenerative disease. Each of these methods comprise administering to a human a recombinant retroviral particle preparation as described above, such that a therapeutically efficacious amount of the desired gene product(s) encoded by the gene of interest carried on the vector construct is produced. As used herein, a “therapeutically effective amount” of a gene product expressed from a vector construct according to the invention is an amount that achieves a desired therapeutic benefit in a patient to an extent greater than that observed when the patient was not treated with the gene product. For instance, when the gene product is factor VIII, a “therapeutically effective amount” refers to the amount of factor VIII needed to produce therapeutically beneficial clotting and will thus generally be determined by each patient's attending physician, although serum levels of about 0.2 ng/mL (about 0.1% of “normal” levels) or more will be therapeutically beneficial. When the gene product is an RNA molecule with intrisic biological activity, such an antisense RNA or ribozyme, a “therapeutically effective amount” is an amount sufficient to achieve a clinically relevant change in the patient's condition through reduced expression of the harmful gene product, most often a protein. In a preferred embodiment, the RNA molecule with intrinsic biological activity will be expressed in transduced T cells in molar excess to the targeted RNA molecule. Expression levels of the heterologous and targeted RNAs can be determined by various assays, e.g., by PCR analysis.

Typical dosages for ex vivo treatment of T cells will generally range from about 10⁵ to 10¹² infectious recombinant retroviral particles, with dosages of 10⁷ to 10¹⁰ infectious particles being preferred. The exact dosage will depend on the number of T cells needed for the particular clinical indication and whether the further expansion of the transduced and selected T cells is required. Thus, the exact dosage for a particular condition can readily be determined experimentally.

The volume that the high titer preparation of retrovirus is delivered in is preferably not greater than 10% of the culture medium volume of the cell culture. More preferably the volume of the high titer retrovirus preparation is less than 1%, still more preferably less than 0.1%, and still more preferably less than 0.01% of the total cell is culture volume. Additionally, the retrovirus is delivered in a medium that is free of agents that disturb or are toxic to the transduced cells in culture (eg. in an aqueous liquid with a composition similar to that of cell culture medium).

T Cells and Non-Dividing Cells

According to the present invention, T cells and non-dividing (or “non-replicating”) cells, or other cells which are resistant to normal transduction methods, are transduced with high efficiency using recombinant retroviral particles in ex vivo procedures. Such cells are preferably animal cells, particularly human cells. Upon introduction into a patient, the desired gene product(s) encoded by the vector construct carried by the retroviral particles achieve a therapeutic benefit. The transduced cells administered to a patient are preferably allogeneic cells, with autologous cells being particularly preferred.

Various techniques may be employed to separate the cells by initially removing cells of dedicated lineage (“lineage-committed” cells). Monoclonal antibodies and monoclonal antibody fragments are particularly useful for identifying markers associated with particular cell lineages and/or stages of differentiation. The antibodies (or antibody fragments) may be attached to a solid support to allow for crude separation. The separation techniques employed should maximize the viability of the fraction to be collected.

### EXAMPLES

The following examples are included to more fully illustrate the present invention. Additionally, these examples provide preferred embodiments of the invention and are not meant to limit the scope thereof. Standard methods for many of the procedures described in the following examples, or suitable alternative procedures, are provided in widely reorganized manuals of molecular biology, such as, for example “Molecular Cloning,” Second Edition (Sambrook, et al., Cold Spring Harbor Laboratory Press, 1987) and “Current Protocols in Molecular Biology” (Ausubel, et al., eds. Greene Associates/Wiley Interscience, NY, 1990).

### Example 1

### Preparation of T Cells for Transduction

Human leukocyte cell lines were grown in RPMI media supplemented with 20% fetal calf serum; penn/strep; NEAA and L-glu. Cell were grown until they were at a density of approximately 5×10⁵ cells/ml and diluted to 1×10⁵/ml. Cells were tranduced in 2 ml volume containing 8 ug/ml polybrene and vector added at the moi's indicated. Four to five days later, cells were pelleted and washed in PBS. For luciferase assays, cells were lysed and assayed according to manufacturer's instructions (Tropix Inc., Bedford, Mass.). Beta-gal vector-tranduced cells were analyzed using the X-gal assay (Nolan et al., 19XX).

A. Transduction of Human Leukocytes by High Titer Retroviral Vectors

Various leukocyte cell lines were tested for functional truncation (i.e., gene expression) with retroviral vectors of varying tropisms. Among those tested were β-gal vectors from two different amphotropic and xenotropic producer cell lines of canine (DA; DX; CFA) and human origin (2×), respectively, [DA/CBβ gal(V); CFA/ND7(V); DX/ND7(V); 2×/CBβ gal(V)] as well as G-pseudotyped CBβ-gal(V) (G-β gal) generated from human 293 2-3 cells. The ampho and xeno vectors were tested at the same titer, all diluted to 1×10⁸ bfu/ml; moi=10, whereas the G-vector was used at 10-fold lower concentration, 10⁷ bfu/ml; moi=1 (bfu=blue cell forming unit and moi=multiplicity of infection). The frequency of blue cells in each transduced culture is summarized below.

In vitro transduction of leukocyte cell lines with vectors of varying tropisms

| Cell line | Cell Type | DA/β gal | CFA/ND7 | DX/ND7 | 2X/β gal | G-β gal |
|---|---|---|---|---|---|---|
| Raji | Burkitts lymphoma | + | + | ++ | + | +/− |
| HL-60 | Promyleocyte | − | +/− | +/− | +/− | − |
| SupT1 | T-cell lymphoma | ++ | ++ | + | ++ | ++ |
| K 562 | Undifferentiated | +++++ | +++++ | ++++ | ++++++ | ++++ |
| | CML | | | | | |
| U 937 | Histiocytic | +/− | + | +/− | + | +/− |
| | lymphoma | | | | | |
| H9 | T-cell lymphoma | +/− | ++ | + | +/− | +/− |
| CEM | T-lymphoblast | + | ++ | + | + | +/− |
| Hut 78 | T-cell lymphoma | +/− | +/− | +/− | +/− | +/− |
| CEM | B/T-cell hybrid | + | ++ | ++++ | +++ | + |
| X174 | | | | | | |

The cell lines were also tested with DA/luci(V), which is a vector preparation encoding the bacterial luciferase gene, for relative gene expression. In this experiment, parallel cultures were spiked with MA virus to see if lack of luciferace expression was at the level of receptor tropism, i.e., would helper virus infect the cells and cause a spread of luci(V) leading to greatly increased expression of luciferase. Cultures were transduced with luci(V) at an moi=5 and MA helper virus at an moi=1. Addition of helper virus to the cultures did not change the luciferase expression profiles, either at the level of bulk protein expression or increase in cellular tropism.

B. Transduction of Primary Cells Using High Titer Retroviral Vectors

Primary murine dendritic cells were transduced using luci(V). The splenic “dendritic cell” fraction consisting of dendritic cells and macrophages was stimulated using GM-CSF and murine splenic B+T-lymphocytes were stimulated using con A. After 24 hours, either β-gal(V) or luci(V) was added at an moi=10. The results are shown below in relative light units.

| Cells | β-gal(V) | luci(V) |
|---|---|---|
| DC | 250 | 2500 |
| DC | 280 | 3100 |
| B + T | 300 | 310 |

These results demonstrate that the splenic dendritic fraction was transduced by high titer amphotropic retroviral vector.

### Example 2

### Preparation of Retroviral Vector Backbones

The following example describes the production of three retroviral vector backbones, designated KT-1, KT-3B, KT-3C. Vector KT-1 differs from KT-3B and KT-3C in that the former lacks a selectable marker which in KT-3B is neomycin resistance, whereas KT-3C confers phleomycin resistance.

The Moloney murine leukemia virus (MoMLV) 5′ long terminal repeat (LTR) EcoR I-EcoR I fragment, including gag sequences, from the N2 vector (Armentano et al., J. Vir. 61:1647, 1987; Eglitas et al., Science 230:1395, 1985) is ligated into the plasmid SK⁺ (Stratagene, La Jolla, Calif.). The resulting construct is designated N2R5. The N2R5 construct is mutated by site-directed in vitro mutagenesis to change the ATG start codon to ATT preventing gag expression. This mutagenized Eminent is 200 base pairs (bp) in length and flanked by Pst I restriction sites. The Pst I-Pst I mutated fragment is purified from the SK⁺ plasmid and inserted into the Pst I site of N2 MoMLV 5′ LTR in plasmid pUC31 to replace the non-mutated 200 bp fragment. The plasmid pUC31 is derived from pUC19 (Stratagene, La Jolla, Calif.) in which additional restriction sites Xho I, Bgl II, BssH II and Nco I are inserted between the EcoR I and Sac I sites of the polylinker. This construct is designated pUC31/N2R5 gM.

A 1.0 kilobase (Kb) MoMLV 3′ LTR EcoR I-EcoR I fragment from N2 is cloned into plasmid SK⁺ resulting in a construct designated N2R3⁻. A 1.0 Kb Cla I-Hind III fragment is purified from this construct.

The Cla I-Cla I dominant selectable marker gene fragment from pAFVXM retroviral vector (Kriegler et al., Cell 38:483, 1984; St. Louis et al., PNAS 85:3150, 1988), comprising a SV40 early promoter driving expression of the neomycin (neo) phosphotransferase gene, is cloned into the SK⁺ plasmid. This construct is designated SK⁺ SV₂-neo A 1.3 Kb Cla I-BstB I gene fragment is purified from the SK⁺ SV₂-neo plasmid.

KT-3B or KT-1 vectors are constructed by a three part ligation in which the Xho I-Cla I fragment containing the gene of interest and the 1.0 Kb MoMLV 3′ LTR Cla I-Hind III fragment are inserted into the Xho I-Hind III site of pUC31/N2R5 gM plasmid. This gives a vector designated as having the KT-1 backbone. The 1.3 Kb Cla I-BstB I neo gene fragment from the pAFVXM retroviral vector is then inserted into the Cla I site of this plasmid in the sense orientation to yield a vector designated as having the KT-3B backbone.

An alternative selectable marker, phleomycin resistance (Mulsant, et al., Som. Cell and Mol. Gen., 14:243, 1988, available from Cayla, Cedex, FR) is used to make the retroviral backbone KT-3C as follows. The plasmid pUT507 (Mulsant, et al., supra) is digested with Nde I and the ends blunted with Klenow polymerase I. The sample is then further digested with Hpa I, Cla I linkers ligated to the mix of fragments, followed by digestion with Cla I to remove excess Cla I linkers. The 1.2 Kb Cla I fragment carrying the RSV LTR and the phleomycin resistance gene is isolated by agarose gel electrophoresis followed by purification using Gene Clean (Bio101, San Diego, Calif.). This fragment is used in place of the 1.3 Kb Cla I-BstB I neomycin resistance fragment to give the backbone KT-3C.

### Example 3

### Preparation of Retroviral Vector Constructs Encoding Proteins

The following example describes the preparation of various retroviral vector constructs encoding different human genes of interest. More specifically, part (A) describes the production of a vector construct encoding the marker gene β galactosidase from E. coli, part (B) human interferon (hIFN), part (C) a retroviral vector construct encoding human interleukin-2 (hIL-2), and part (D) the production of two retroviral vector constructs coding for human factor VIII. The first factor VIII construct, codes for the B domain deleted form of the protein, while the second construct codes for full length factor VIII.

A. Preparation of β-Gal Vectors

pCBβ-gal is prepared as described in Irusin et al. (1994) J. Virol. pND7 is obtained by inserting the E. coli β-gal into the pND5 (se below) vector after excision of the Factor VIII gene.

B. Preparation of KT-rhγ-IFN

To obtain the human γ-IFN gene, the murine homologue is first cloned as follows: A mγ-IFN cDNA is cloned into the EcoR I site of pUC1813 essentially as set forth below. Briefly, pUC1813 (containing a sequence encoding γ-IFN) is prepared as essentially described by Kay et al., (Nucleic Acids Research 15:2778, 1987; and Gray et al., PNAS 80:5842, 1983). The mγ-IFN cDNA is retrieved by EcoR I digestion of pUC 1813, and the isolated fragment is cloned into the EcoR I site of phosphatase-treated pSP73 (Promega; Madison, Wis.). This construct is designated SP mγ-IFN. The orientation of the cDNA is verified by appropriate restriction enzyme digestion and DNA sequencing. In the sense orientation, the 5′ end of the cDNA is adjacent to the Xho I site of the pSP73 polylinker and the 3′ end adjacent to the Cla I site. The Xho I-Cla I fragment containing the mγ-IFN cDNA in either sense or antisense orientation is retrieved from SP mγ-IFN construct and cloned into the Xho I-Cla I site of the KT-3 retroviral backbone. This construct is designated KT mγ-IFN.

1. Preparation of Sequences Encoding Hγ-IFN Utilizing PCR

(a) PHA Stimulation of Jurkat Cells

Jurkat cells (T cell line ATCC No. CRL 8163) are resuspended at a concentration of 1×10⁶ cells/ml in RPMI growth media (Irvine Scientific; Santa Ana, Calif.) with 5% fetal bovine serum (FBS) to a final volume of 158.0 ml. Phytochemoagglutinin (“PHA”) (Curtis Mathes Scientific, Houston, Tex.) is added to the suspension to a final concentration of 1%. The suspension is incubated at 37° C. in 5% CO₂ overnight. The cells are harvested on the following day and aliquoted into three 50.0 ml centrifuge tubes. The three pellets are combined in 50 ml 1×phosphate buffered saline (PBS, 145 mM, pH 7.0) and centrifuged at 1000 rpm for 5 minutes. The supernatant is decanted and the cells are washed with 50.0 ml PBS. The cells are collected for RNA isolation.

(b) RNA Isolation

The PHA stimulated Jurkat cells are resuspended in 22.0 ml guanidinium solution (4 M guanidinium thiocyanate; 20 mM sodium acetate, pH 5.2; 0.1 M dithiothreitol, 0.5% sarcosyl). This cell-guanidinium suspension is then passed through a 20 gauge needle six times in order to disrupt cell membranes. A CsCl solution (5.7 M CsCl, 0.1 M EDTA) is then overlaid with 11.0 mL of the disrupted cell-guanidinium solution. The solution is centrifuged for 24 hours at 28,000 rpm in a SW28.1 rotor (Beckman, Fullerton, Calif.) at 20° C. After centrifugation the supernatant is carefully aspirated and the tubes blotted dry. The pellet is resuspended in a guanidinium-HCl solution (7.4 M guanidinium-HCl; 25 mM Tris-HCl, pH 7.5; 5 mM dithiothreitol) to a final volume of 500.0 μl. This solution is transferred to a microcentrifuge tube. Twelve and one-half microliters of concentrated Glacial acetic acid (HAc) and 250 μl of 100% EtOH are added to the microfuge tube. The solution is mixed and stored for several days at −20° C. to precipitate RNA.

After storage, the solution is centrifuged for 20 minutes at 14,000 rpm, 4° C. The pellet is then resuspended in 75% ETOH and centrifuged for 10 minutes in a microfuge at 14,000 rpm, 4° C. The pellet is dried by centrifugation under vacuum, and resuspended in 300 L deionized (DI) H₂O. The concentration and purity of the RNA is determined by measuring optical densities at 260 and 280 nm.

(c) Reverse Transcription Reaction

Immediately before use, 5.0 1 (3.4 mg/mL) of purified Jurkat RNA is heat treated for 5 minutes at 90° C., and then placed on ice. A solution of 10.0 μl of 10×PCR buffer (500 mM KCl; 200 mM Tris-HCl, pH 8.4; 25 mM MgCl₂; 1 mg/ml bovine serum albumin (BSA)); 10.0 μl of 10 mM dATP, 10.0 μl of 10 mM dGTP, 10.0 μl of 10 mM dCTP, 10.0 μl of 10 mM dTTP, 2.5 μl RNasin (40,000 U/ml, Promega: Madison Wis.) and 33.0 μl DI H₂O, is added to the heat treated Jurkat cell RNA. To this solution 5.0 μl (10⁸ nmol/mL) (Sequence ID No. 1), and 5.0 μl (200,000 U/ml) MoMLV reverse transcriptase (Bethesda Research Laboratories, EC 3.127.5, MD) is mixed in a microfuge tube and incubated at room temperature for 10 minutes. Following the room temperature incubation, the reaction mixture is incubated for 1 hour at 37° C., and then incubated for 5 minutes at 95° C. The reverse transcription reaction mixture is then placed on ice in preparation for PCR.

(d) PCR Amplification

The PCR reaction mixture contains 100.0 μl reverse transcription reaction; 356.01 DI H₂O; 40.0 μl 10×PCR buffer; 1.0 μl (137 nmol/mL) V-OLI #5 (Sequence ID No. 2); 0.5 μl (320 nmol/mL) V-OLI #6 (Sequence ID No. 3), and 2.5 μl, 5,000 U/ml, Taq polymerase (EC 2.7.7.7, Perkin-Elmer Cetus, Calif.). One hundred microliters of this mixture is aliquoted into each of 5 tubes.

Sequence ID No. 1

5′-3′: TAA TAA ATA GAT TTA GAT TTA

This primer is complementary to a sequence of the mγ-IFN cDNA 30 base pairs downstream of the stop codon.

V (Sequence ID No. 2)

5′-3′: GC CTC GAG ACG ATG AAA TAT ACA AGT TAT ATC TTG

This primer is complementary to the 5′ coding region of the mγ-IFN gene, beginning at the ATG start codon. The 5′ end of the primer contains a Xho I restriction site.

Sequence ID No. 3

5′-3′: GA ATC GAT CCA TTA CTG GGA TGC TCT TCG ACC TGG

This primer is complementary to the 3′ coding region of the mγ-IFN gene, ending at the TAA stop codon. The 5′ end of the primer contains a Cla I restriction site.

Each tube was overlaid with 100.0 μl mineral oil, and placed into a PCR machine (Ericomp Twin Block System, Ericomp, Calif.). The PCR program regulates the temperature of the reaction vessel f at 95° for 1 minute, next at 67° for 2 minutes and finally at 72° for 2 minutes. This cycle is repeated 40 times. The last cycle regulates the temperature of the reaction vessel first at 95° for 1 minute, next at 67° for 2 minutes and finally at 72° for 7 minutes. The completed PCR amplification reactions are stored at 4° for 1 month in preparation for PCR DNA isolation.

(e) Isolation of PCR DNA

The aqueous phase from the PCR amplification reactions are transferred into a single microfuge tube. Fifty microliters of 3 M sodium acetate and 500.0 μl of chloroform:isoamyl alcohol (24:1) is added to the solution. The solution is vortexed and then centrifuged at 14,000 rpm at room temperature for 5 minutes. The upper aqueous phase is transferred to a fresh microfuge tube and 1.0 mL of 100% EtOH is added. This solution is incubated for 4.5 hours at −20° C. and then centrifuged at 14,000 rpm for 20 minutes. The supernatant is decanted, and the pellet is rinsed with 500.0 μl of 70% EtOH. The pellet is dried by centrifugation under a vacuum. The isolated hγ-IFN PCR DNA is resuspended in 10.0 μl DI H₂O.

2. Construction of h-IFN Retroviral Vectors

(a) Creation and Isolation of Blunt-Ended hg-IFN PCR DNA Fragments

The hγ-INF PCR DNA is blunt ended using T4 DNA polymerase. Specifically, 10.0 μl of PCR amplified DNA; 2.0 μl, 10×, T4 DNA polymerase buffer (033 M Tris-acetate, pH 7.9, 0.66 M potassium acetate, 0.10 M magnesium acetate, 5 mM dithiothreitol, 1 mg/mL bovine serum albumin (BSA)); 1.0 μl, 2.5 mM dNTP (a mixture containing equal molar concentrations of dATP, dGTP, dTTP and dCTP); 7.0 μl DI H₂O; 1.0 μl, 5000 U/mL, Klenow fragment (EC 2.7.7.7, New England Biolabs, Mass.); and 1.0 μl, 3000 U/ml, T4 DNA polymerase (EC 2.7.7.7, New England Biolabs, Mass.) are mixed together and incubated at 37° C. for 15 minutes. The reaction mixture is then incubated at room temperature for 40 minutes and followed by an incubation at 68° C. for 15 minutes.

The blunt ended hγ-INF is isolated by agarose gel electrophoresis. Specifically, 2.0 μl of loading dye (0.25% bromophenol blue; 0.25% xylene cyanol; and 50% glycerol) is added to reaction mixture and 4.0 μl is loaded into each of 5 lanes of a 1% agarose/Tris-borate-EDTA (TBE) gel containing ethidiurn bromide. Elecrophoresis of the gel is performed for 1 hour at 100 volts. The desired DNA band containing hγ-INF, approximately 500 base pairs in length, is visualized under ultraviolet light.

This band is removed from the gel by electrophoretic transfer onto NA 45 paper (Schleicher and Schuell, Keene, N.H. The paper is incubated at 68° C. for 40 minutes in 400.0 μl of high salt NET buffer (1 M NaCl; 0.1 mM EDTA; and 20 mM Tris, pH 8.0) to elute the DNA. The NA 45 paper is removed from solution and 400.0 μl of phenol:chloroform:isoamyl alcohol (25:24:1) is added. The solution is vortexed and centrifuged at 14,000 for 5 minutes. The upper aqueous phase is transferred to a fresh tube and 400.0 μl of chloroform:isoamyl alcohol (24:1) is added. The mixture is vortexed and centrifuged for 5 minutes. The upper aqueous phase is transferred, a second time, to a fresh tube and 700.0 μl of 100% EtOH is added. The tube is incubated at −20° C. for 3 days. Following incubation, the DNA is precipitated from the tube by centrifugation for 20 minutes at 14,000 rpm. The supernatant is decanted and the pellet is rinsed with 500.0 μl of 70% EtOH. The pellet containing blunt ended hγ-IFN DNA, is dried by centrifugation under vacuum and resuspended in 50.0 μl of DI H₂O.

The isolated blunt ended hγ-IFN DNA is phosphorylated using polynucleotide kinase. Specifically, 25.0 μl of blunt-ended hγ-IFN DNA, 3.0 μl of 10×kinase buffer (0.5 M Tris-HCl, pH 7.6; 0.1 M MaCl₂; 50 mM dithiothreitol; 1 mM spermidine; 1 mM EDTA), 3.0 μl of 10 mM ATP, and 1.0 μl of T4 polynucleotide kinase (10,000 U/ml, EC 2.7.1.78, New England Biolabs, MD) is mixed and incubated at 37° C. for 1 hour 45 minutes. The enzyme is then heat inactivated by incubating at 68° C. for 30 minutes.

(b) Ligation of hγ-IFN PCR DNA Into the SK⁺ Vector

An SK⁺ plasmid is digested with Hinc II restriction endonuclease and purified by agarose gel electrophoresis as described below. Specifically, 5.9 μl (1.7 mg/mL) SK⁺ plasmid DNA (Stratagene; San Diego, Calif.); 4.0 μl 10×Universal buffer (Staagene, San Diego, Calif.); 30.1 μl DI H₂O, and 4.0 μl Hinc II, 10,000 U/mL, are mixed in a tube and incubated for 7 hours at 37° C. Following incubation, 4.0 μl of loading dye is added to the reaction mixture and 4.0 μl of this solution is added to each of 5 lanes of a 1% agarose/TBE gel containing ethidium bromide. Electrophoresis of the gel is performed for 2 hours at 105 volts. The Hinc II cut SK⁻ plasmid, 2958 base pairs in length, is visualized with ultraviolet light. The digested SK⁺ plasmid is isolated by gel electrophoresis.

Dephosphorylation of the Hinc II cleavage site of the plasmid is performed using calf intestine alkaline phosphatase. Specifically, 50.0 μl digested SK⁺ plasmid; 5.0 μl 1 M Tris, pH 8.0; 2.0 μl 5 mM EDTA, pH 8.0; 43.0 μl H₂O and 2.0 μl, 1,000 U/mL, calf intestinal phosphatase (“CIP”) (Boehringer Mannheim, Indianapolis, Ind.) are mixed in a tube and incubated at 37° C. for 15 minutes. Following incubation, 2.0 μl CIP is added, and the solution is incubated at 55° C. for 90 minutes. Following this incubation, 2.5 μl 20% sodium dodecyl sulfate (“SDS”), 1.0 μl 0.5 M EDTA, pH 8.0, and 0.5 μl, 20 mg/mL, proteinase K (EC 3.4.21.14, Boehringer Mannheim, Indianapolis, Ind.) are added, and the solution is incubated at 55° C. for 2 hours. This solution is cooled to room temperature, and 110.0 μl phenol:chloroform:isoamyl alcohol (25:24:1) is added. The mixture is vortexed and centrifuged at 14,000 rpm for 5 minutes. The upper aqueous phase is transferred to a fresh tube and 200.0 μl of 100% EtOH is added. This mixture is incubated at 70° C. for 15 minutes. The tube is centrifuged and the pellet is rinsed with 500.0 μl of 70% EtOH. The pellet was then dried by centriffiation under a vacuum. The dephosphorylated SK⁺ plasmid is resuspended in 40 μl DI H₂O.

The hγ-INF PCR DNA is ligated into the SK⁺ plasmid using T4 DNA ligase. Specifically, 30.0 μl blunt ended, phosphorylated, hγ-IFN PCR DNA reaction mixture, 2.0 μl dephosphorylated SK⁺ plasmid and 1.0 μl T4 DNA ligase are combined in a tube and incubated overnight at 16° C. DNA was isolated using a minprep procedure. More specifically, the bacterial strain DH5a (Gibco BRL, Gaithersburg, Md.) is transformed with 15.0 μl of ligation reaction mixture, plated on Luria-Bertani agar plates (LB plates) containing ampicillin and 5-bromo4chloro-3-indolyl-β-D-galactoside (X-gal, Gold Biotechnology; St. Louis, Mo.), and incubated overnight at 37° C. DNA is isolated from white bacterial colonies using the procedure described by Sambrook et al. (Molecular Cloning, Cold Springs Harbor Press, 1989). The presence of the hγ-IFN gene is determined by restriction endonuclease cleavage with Xho I, Cla I, Ava II, Dra I, and Ssp I. The expected endonuclease restriction cleavage fragment sizes for plasmids containing the hγ-IFN gene are presented in Table 2. The isolated DNA plasmid is designated SK hγ-IFN and used in constructing the retroviral vectors.

TABLE 2

| Enzyme | Fragment Size (bp) |
|---|---|
| Xho I and Cla I | 500, 2958 |
| Ava II | 222, 1307, 1937 |
| Dra I | 700, 1149, 1500 |
| Ssp I | 750, 1296, 2600 |

(c) Ligation of hγ-IFN Gene Into Retroviral Vector

The interferon gene is removed from SK hγ-IFN vector by digestion with Xho I and Cla I restriction endonucleases. The resulting fragment containing the hγ-IFN gene is approximately 500 bp in length and is isolated in a 1% agarose/TBE gel electrophoresis. The Xho I-Cla I hγ-IFN fragment is then ligated into the KT-3 retroviral backbone. This construct is designated KT hγ-IFN. The structure and presence expression of hγ-IFN is determined by transforming DH5a bacterial strain with the KT hγ-IFN construct. Specifically, the bacteria is transformed with 15.0 μl of ligation reaction mixture. The transformed bacterial cells are plated on LB plates containing ampicillin. The plates are incubated overnight at 37° C. and bacterial colonies are selected. The DNA is isolated as described in (b) above, and digested with Xho I, Cla I, Dra I, Nde I, and Ssp I. The expected endonuclease restriction cleavage fragment sizes for plasmids containing the hγ-IFN gene are presented in Table 3.

TABLE 3

| Enzyme | Fragment Size (bp) |
|---|---|
| Xho I and Cla I | 500, 6500 |
| Nde I | 1900, 5100 |
| Dra I | 692, 2700, 3600 |
| Ssp I | 541, 1700, 4700 |

Subsequent sequencing of KT hγ-IFN, the retroviral vector, revealed the presence of a one base pair deletion within the hγ-IFN gene. This deletion is reversed using multi-step PCR procedure.

i. Sequence Selection

Sequences are obtained from IBI Pustell sequence analysis program (Int. Biotech, Inc., New Haven, Conn.).

The following hγ-IFN primer sequences are used:

Sequence ID No. 4

5′-3′: G CCT CGA GCT CGA GCG ATG AAA TAT ACA AGT TAT ATC TTG

This primer is the sense sequence complimentary to the start codon ATG region extending seven codons upstream of hγ-IFN gene, and is designated hγ-IFN 1b.

Sequence ID No. 5

5′-3′: GTC ATC TCG TTT CTT TTT GTT GCT ATT

This primer is the anti-sense sequence complimentary to codons 106 to 120 of the hγ-IFN gene, and is designated hγ-IFN Rep B.

Sequence ID No. 6

5′-3′: AAT AGC AAC AAA AAG AAA CGA GAT GAC

This primer is the sense sequence complimentary to codons 106 to 120 of the hγ-IFN gene, and is designated hγ-IFN Rep A.

Sequence ID No. 7

5′-3′: G CAT CGA TAT CGA TCA TTA CTG GGA TGC TCT TCG ACC TCG

This primer is the anti-sense sequence complimentary to the stop codon ATT region and extending seven codons upstream of the hγ-IFN gene, and is designated hγ-IFN 3b.

ii. Initial PCR

A solution of 1×10⁶ KT hγ-IFN plasmid molecules in 398.0 μl, DI H₂O; 50 μl, 10×PCR buffer (500 mM KCl and 200 mM Tris-HCl, pH 8.4; 25 mM MgCl₂; 1.0 mg/ml BSA); 5.0 μl, 2.5 mM dATP; 5.0 μl, 2.5 mM dGTP; 5.0 μl, 2.5 mM dCTP; 5.0 μl, 2.5 mM dTTP; 12.0 μl, 18.6 nmol/ml, oligonucleotide hγ-IFN 1b; 15.0 μl, 24.6 nmol/ml, oligonucleotide hγ-IFN RepB; and 2.5 μl, Taq polymerase is mixed in a microfuge tube and 50 μl is aliquoted into 10 tubes. Similarly, a solution of 1×10⁶ KT hγ-IFN plasmid molecules in 395.0 μl, DI H₂O; 50.0 μl, 10×PCR buffer (500 mM KCl; 200 mM Tris-HCl, pH 8.4; 25 mM MgCl₂; 1 mg/ml BSA); 5.0 μl, 2.5 mM dATP; 5.0 μl, 2.5 mM dGTP; 5.0 μl, 2.5 mM dCTP; 5.0 μl, 2.5 mM dTTP; 13 μl, 23.4 nmol/ml, oligonucleotide hγ-IFN RepA; 17.0 1 μl, 18.0 nmol/ml, oligonucleotide hγ-IFN 3b; and 2.5 μl Taq polymerase is mixed in a microfuge tube and 50.0 μl is aliquoted into 10 tubes. The 20 tubes are placed in a PCR machine (Model 9600, Perkin Elmer Cetus; Los Angeles, Calif.). The PCR program regulates the temperature of the reacton vessel in the first cycle at 94° C. for 2 minutes. The next 35 cycles are regulated at 94° C. for 0.5 minutes, then at 55° C. for 0.5 minutes and finally at 72° C. for 1 minute. The final cycle is regulated at 72° C. for 10 minutes. This cycling program is designated Program 10.

Following PCR amplification, 225.0 μl of each reaction tube is mixed with 25.0 μl loading dye (0.25% bromophenol blue, 0.25% xylene cyanol and 50% glycerol, agarose gel loading dye) and loaded into the wells of a 2% agarose gel containing ethidium bromide. The gel is electrophoresed at approximately 90 volts for 1 hour. Ultraviolet light is used to visualize the DNA band separation. Two bands are isolated, one fragment of 250 bp in size and the other of 150 bp in size by electrophoretic transfer onto NA 45 paper. Following precipitation, each of the two DNA pellets is resuspended in 20.01 DI H₂O and prepared for further PCR amplification.

iii. Annealing and Second Round PCR

A solution of 20.0 μl of the 150 bp PCR DNA; 20.0 μl of the 350 bp PCR DNA: 161.5 μl, DI H₂O; 25.0 μl, 10×PCR buffer (500 mM KCl; 200 mM Tris-HCl, pH 8.4; 25 mM MgCl₂; and 1 mg/ml BSA); 2.5 μl, 2.5 mM dATP; 2.5 μl, 2.5 mM dGTP; 2.5 μl, 2.5 mM dCTP; 2.5 μl, 2.5 mM dTTP; and 1.25 μl Taq polymerase is mixed in a microfuge tube and 47.3 μl aliquoted into each of 5 tubes. Each tube is placed in a PCR machine (Model 9600, Perkin-Elmer-Cetus, CA). The PCR program regulates the temperature of the reaction vessel for 5 cycles at 94° C. for 0.5 minutes. The next cycle is regulated at 55° C. for 1 minute. Following this cycle, 1.2 μl hγ-IFN 1b and 1.5 μl hγ-IFN 3b are added to the reaction mixture. The tubes are then PCR amplified using program 10. The product is designated rhγ-IFN.

iv. Creation and Isolation of Blunt-Ended rhg-IFN PCR DNA Fragment

The PCR amplified hγ-IFN DNA is blunt ended using T4 polymerase. Specifically, 120.0 μl rhγ-IFN PCR solution is mixed with 125 μl, 2.5 mM dATP; 1.25 μl, 2.5 mM dGTP; 1.25 μl, 2.5 mM dCTP; 1.25 l, 2.5 mM dTTP; 1 l, T4 DNA polymerase; and 1.0 μl Klenow fragment. This mixture is incubated at room temperature for 10 minutes. Following incubation, 13.0 μl of agarose gel loading dye is added to the mixture and this solution is loaded into a 1% agarose gel. The gel is electrophoresed at approximately 90 volts for 1 hour. Ultraviolet light is used to visualize the DNA banding. A 500 bp band is isolated by electrophoretic transfer onto NA 45 paper as described above. Following precipitation, the DNA pellet is reuspended in 12.0 l DI H₂O.

The isolated 500 bp fragment is blunt ended using T4 polynucleotide kinase. Specifically, 1.0 mg of this fragment is mixed with 1.5 μl 10×kinase buffer (0.5 mM Tris-HCl, pH 7.6; 0.1 mM MgCl₂; 50 mM dithiothreitrol; 1 mM spermidine; 1 mM EDTA); 1.5 μl, 10 mM ATP; and 1.0 μl, T4 polynucleotide kinase, and incubated at 37° C. for 30 minutes.

v. Ligation of rhγ-IFN PCR DNA Into the SK⁺ Vector

The rhγ-IFN PCR DNA is ligated into the SK⁺ vector. A solution of 2.0 μl hγ-IFN PCR DNA-kinase reaction mixture; 2.0 μl CIP treated SK⁺ vector; and 1.0 μl, T4 DNA ligase is incubated at 16° C. for 4 hours. DH5a bacteria is transformed as described above.

vi. Ligation of hγ-IFN Gene Into Retroviral Vector

Ligation of hγ-IFN gene into retroviral vector is performed as described above. The new vector is designated KT hγ-IFN.

C. Preparation of KT-hIL-2

The method for cloning hIL-2 into KT-3 retroviral vector is essentially identical to the procedure for cloning hg-IFN into KT-3, with the exception that different primers are required for amplifcation of the hIL-2 DNA sequence. The following hIL-2 PCR primer sequences are used:

V-OLI #55 (Sequence ID No. 8)

5′-3′: ATA AAT AGA AGG CCT GAT ATG

This primer is complimentary to a sequence of the hIL-2 cDNA downstream of the stop codon.

V-OLI #1 (Sequence ID No. 9)

5′-3′: GC CTC GAG ACA ATG TAC AGG ATG CAA CTC CTG TCT

This primer is the sense sequence of the hIL-2 gene complimentary to the 5′ coding region beginning at the ATG start codon. The 5′ end of the primer contains a Xho I restriction site.

V-OLI #2 (Sequence ID No. 10)

5′-3′: GA ATC GAT TTA TCA AGT CAG TGT TGA GAT GAT GCT

The primer is the anti-sense sequence of the hIL-2 gene complimentary to the 3′ coding region ending at the TAA stop codon. The 5′ end of the primer contains the Cla I restricton site.

D. Preparation of Factor VIII Vectors

The following is a description of the construction of several retroviral vectors encoding factor VIII. Due to the size of the full length factor VIII gene (7,056 bp), packaging constraints of retroviral vectors and because selection for transduced cells is not a requirement for ex vivo hematopoietic stem cell therapy, a retroviral backbone, e.g., KT-1, lacking a selectable marker gene is employed.

A gene encoding full length factor VIII can be obtained from a variety of sources. One such source is the plasmid pCIS-F8 (EP 0 260 148 A2, published Mar. 3, 1993), which contains a full length factor VIII cDNA whose expression is under the control of a CMV major immediate-early (CMV MIE) promoter and enhancer. The factor VIII cDNA contains approximately 80 bp of 5′ untranslated sequence from the factor VIII gene and a 3′ untranslated region of about 500 bp. In addition, between the CMV promoter and the factor VIII sequence lies a CMV intron sequence, or “cis” element. The cis element, spanning about 280 bp, comprises a splice donor site from the CMV major immediate-early promoter about 140 bp upstream of a splice acceptor from an immunoglobulin gene, with the intervening region being supplied by an Ig variable region intron.

i. Construction of a Plasmid Encoding Retroviral Vector JW-2

A plasmid, pJW-2, encoding a retroviral vector for expressing full length factor VIII is constructed using the KT-1 backbone from pKT-1. To facilitate directional cloning of the factor VIII cDNA insert into pKT-1, the unique Xho I site is converted to a Not I site by site directed mutagenesis. The resultant plasmid vector is then opened with Not I and Cla I. pCIS-F8 is digested to completion with Cla I and Eag I, for which there are two sites, to release the fragment encoding full length factor VIII. This fragment is then ligated into the Not I/Cla I restricted vector to generate a plasmid designated pJW-2.

i. Construction of a Plasmid Encoding Retroviral Vector ND-5

A plasmid vector encoding a truncation of about 80% (approximately 370 bp) of the 3′ untranslated region of the factor VIII cDNA, designated pND-5. is constructed in a pKT-1 vector as follows: As described for pJW-2, the pKT-1 vector employed has its Xho I restriction site replaced by that for Not I. The factor VIII insert is generated by digesting pCIS-F8 with Cla I and Xba I, the latter enzyme cutting 5′ of the factor VIII stop codon. The approximately 7 kb fragment containing all but the 3′ coding region of the factor VIII gene is then purified. pCIS-F8 is also digested with Xba I and Pst I to release a 121 bp fragment containing the gene's termination codon. This fragment is also purified and then ligated in a three way ligation with the larger fragment encoding the rest of the factor VIII gene and Cla I/Pst I restricted BLUESCRIPT® KS⁺ plasmid (Stratagene, San Diego, Calif.) to produce a plasmid designated pND-2.

The unique Sma I site in pND-2 is then changed to a Cla I site by ligating Cla I linkers (New England Biolabs, Beverly, Mass.) under dilute conditions to the blunt ends created by a Sma I digest. After recircularization and ligation, plasmids containing two Cla I sites are identified and designated pND-3.

The factor VIII sequence in pND-3, bounded by Cla I sites and containing the full length gene with a truncation of much of the 3′ untranslated region, is cloned as follows into a plasmid backbone derived from a Not I/Cla I digest of pJW-1 [a pKT-1 derivative by cutting at the Xho I site, blunting with Klenow, and inserting a Not I linker (New England Biolabs)], which yields a 5.2 kb Not I/Cla I fragment. pCIS-F8 is cleaved with Eag I and Eco RV and the resulting fragment of about 4.2 kb, encoding the 5′ portion of the full length factor VIII gene, is isolated. pND-3 is digested with Eco RV and Cla I and a 3.1 kb fragment is isolated. The two fragments containing portions of the factor VIII gene are then ligated into the Not I/Cla I digested vector backbone to produce a plasmid designated pND-5.

iii. Construction of a B Domain-Deleted Factor VIII Vector

The precursor DNA for the B-deleted FVIII is obtained from Miles Laboratory. This expression vector is designated p25D and has the exact backbone as pCISF8 above. The Hpa I site at the 3′ of the FVII8 cDNA in p25D is modified to Cla-I by oligolinkers. An Acc I to Cla I fragment is clipped out from the modified p25D plasmid. This fragment spans the B-domain deletion and includes the entire 3′ two-thirds of the cDNA. An Acc I to Cla I fragment is removed from the pJW-2 above, and replaced with the modified B-domain deleted fragment just described. This construct is designated B-del-1.

As those in the art will appreciate, after construction of plasmids encoding retroviral vectors such as those described above, such plasmids can then be used in the production of various cell lines from which infectious recombinant retroviruses can be produced.

### Example 4

### Packaging Cell Production

A. MLV Structural Gene Expression Vectors

To decrease the possibility of replication-competent virus being generated by genetic interactions between the MLV proviral vector DNA and the structural genes of the packaging cell line (“PCL”), separate expression vectors, each lacking the viral LTR, were generated to express the gag/pol and env genes independently. To further decrease the possibility of homologous recombination with MLV vectors and the resultant generation of replication-complement virus, minimal sequences other than the protein coding sequences were used. In order to express high levels of the MLV structural proteins in the host cells, strong transcriptional promoters (CMV early and Ad5 major late promoters) were utilized. An example of the construction of a MoMLV gag/pol expression vector pSCV10 follows:

1. The 0.7 Kb HinCII/XmaIII fragment encompassing the human cytomegalovirus (CMV) early transcriptional promoter (Boshart, et al., Cell 41:521, 1985) was isolated.

2. A 5.3 Kb PstI(partial)/ScaI fragment from the MoMLV proviral plasmid, MLV-K (Miller, et al., Mol. Cell Biol. 5:531, 1985) encompassing the entire gag/pol coding region was isolated.

3. A 0.35 Kb DraI fragment from SV40 DNA (residues 2717-2363) encompassing the SV40 late transcriptional termination signal was isolated.

4. Using linkers and other standard recombinant DNA techniques, the CMV promoter-MoMLV gag/pol-SV40 termination signal was ligated into the bluescript vector SK⁺ (Stratagene, San Diego, Calif.).

An example of the construction of an MLV xenotropic envelope expression vector follows.

1. A 2.2 Kb NaeI/NheI fragment containing the coding region of the xenotropic envelope obtained from clone NZB9-1 (O'Neill. et al., J. Virol. 53:100, 1985) was isolated.

2. Using linkers and other standard recombinant DNA techniques, the CMV early promoter and SV40 late termination signal described for the gag/pol expression above (pSCV10) were ligated in the order CMV promoter-xeno env-termination signal.

B. Host Cell Selection

Host cell lines were screened for their ability to efficiently (high titer) rescue a drug resistance retroviral vector A alpha N2 (Armentano, et al., J. Vir. 61:1647, 1987; and Eglitas, et al., Science 230:1395, 1985) using replication competent retrovirus to produce the gag/pol and env structural genes (“MA” virus). Titer was measured from confluent monolayers 16 h after a medium change by adding filtered supernatants (0.45 um filters) to 5×10⁴ NIH 3T3 TK⁻ cells on a 6 cm tissue culture plate in the presence of 4 ug/ml polybrene followed by selection in G418. Among the non-murine cell lines which demonstrated the ability to package MoMLV-based vector with high titre were the cell lines CF2 (canine), D17 (canine), 293 (human), and HT1080 (human). These cell lines are preferred for production of packaging and producer cell lines, although many other cells may be tested and selected by such means.

C. Generation of Packaging Cell Lines

(i) Preparation of gag/pol Intermediates

As examples of the generation of gag/pol intermediates for PCL production, D17 (ATCC No. CCL-183), 293 (ATCC No. 1573), and HT1080 (ATCC No. CCL 121) cells were co-transfected with 1 ug of the methotrexate resistance vector, pFR400 (Graham and van der Eb, Virology 52:456, 1973), and 10 ug of the MoMLV gag/pol expression vector, pSCV10 (above) by calcium phosphate co-precipitation (D17 and HT1080, see Graham and van der Eb, supra), or lipofection (293, see Felgner, et al., Proc. Natl. Acad. Sci., USA 84:7413, 1987). After selection for transfected cells in the presence of the drugs dipyrimidol and methotrexate, individual drug resistant cell colonies were expanded and analyzed for MoMLV gag/pol expression by extracellular reverse transcriptase (RT) activity (modified from Goff, et al., J. Virol. 38:239, 1981) and intracellular p30^{gag} by Western blot using anti-p30 antibodies (goat antiserum #77S000087 from the National Cancer Institute). This method identified individual cell clones of each cell type which expressed 10-50×higher levels of both proteins compared with that of the packaging cell line PA317, as shown in Table 4.

TABLE 4
PROPERTIES OF MoMLV GAG/POL-EXPRESSING CELLS

| | | | LARNL |
|---|---|---|---|
| | RT | p30^{gag} | TITRE |
| CELL NAME | ACTIVITY (CPM) | EXPRESSION | (CFU/ML) |
| 3T3 | 800 | − | N.D. |
| PA317 | 1350 | +/− | 1.2 × 10³ |
| D17 | 800 | − | N.D. |
| D17 4-15 | 5000 | +++++ | 1.2 × 10⁴ |
| D17 9020 | 2000 | +++ | 6.0 × 10³ |
| D17 9-9 | 2200 | ++ | 1.0 × 10³ |
| D17 9-16 | 6100 | +++++ | 1.5 × 10⁴ |
| D17 8-7 | 4000 | − | N.D. |
| HT 1080 | 900 | − | N.D. |
| HTSCV21 | 16400 | +++++ | 8.2 × 10³ |
| HTSCV25 | 7900 | +++ | 2.8 × 10³ |
| HTSCV42 | 11600 | ++ | 8.0 × 10² |
| HTSCV26 | 4000 | − | <10 |
| 293 | 600 | − | N.D. |
| 293 2-3 | 6500 | +++++ | 7 × 10⁴ |
| 293 5-2 | 7600 | +++++ | N.D. |

The biological activity of these proteins was tested by introducing a retroviral vector, LARNL which expresses both the amphotropic envelope and a Neo⁺ marker which confers resistance to the drug G418. In every case, co-expression of gag/pol in the cell line and env from the vector allowed efficient packaging of the vector as determined by cell-free transfer of G4 18 resistance to 3T3 cells (titer). Titer was measured from confluent monolayers 16 h after a medium change by adding filtered supernatants (0.45 μm filters) to 5×10⁴ NIH353 TK⁺ cells on a 6 cm tissue culture plate in the presence of 4 ug/ml polybrene followed by selection in G418. Significantly, the vector titers from the cell lines correlated with the levels of p30^{gag} (Table 4). Since the level of env should be the same in each clone and is comparable to the level found in PA317 (data not shown), this indicates that titre was limited by the lower levels of gag/pol in these cells (including PA317). The titre correlated more closely with the levels of p30^{gag} than with the levels of RT.

(ii) Conversion of gag/pol Lines Into Xenotropic Packaging Cell Lines

As examples of the generation of xenotropic PCLs, the gag/pol over-expressors for D17 (4-15) and HT1080 (SCV21) were co-transfected by the same techniques described above except that 1 μg of either the phleomycin resistance vector, pUT507 (for SCV21), or the hygromycin B resistance marker, pY3 (for 4-15, see Blochlinger and Diggelmann, Mol. Cell Biol. 4:2929, 1984), and 10 μg of the xenotropic envelope expression vector, pCMVxeno (above) was used. After selection for transfected cells in the presence of phleomycin or hygromycin, respectively, individual drug resistant cell colonies were expanded and analyzed for intracellular expression of MLV p30^{gag} and gp75^{env} proteins by Western blot using specific antisera. Clones were identified which expressed relatively high levels of both gag/pol and xeno env.

A number of these xenotropic packaging cell lines were tested for their capacity to package retroviral vectors by measuring titre after the introduction of retroviral vectors. The results are presented in Table 5, below.

TABLE 5
VECTOR TITRE ON XENOTROPIC PCLs

| | | KT-1 TITRE (CFU/ML) |
|---|---|---|
| | CELL CLONE | ON HT1080 CELLS |
| HT1080 | SCV21 | 1.0 × 10⁵ |
| | XF1 | 1.0 × 10⁵ |
| | XF7 | 1.0 × 10⁵ |
| | XF12 (HX) | 4.5 × 10⁵ |
| D17 | 4-15 | |
| | X6 | 9.0 × 10⁴ |
| | X10 (DX) | 1.3 × 10⁵ |
| | X23 | 8.0 × 10⁴ |

Highest titers are obtained when retroviral vectors are introduced into packaging cell lines by infection, as opposed to transfection (Miller, et al., Somat. Cell Mol. Genet., 12:175, 1986). However, the xenotropic packaging cell lines described herein are blocked for infection by recombinant xenotropic retroviral particles since the cells express a xenotropic env protein (i.e., “viral interference”). To overcome the problem of “viral interference,” whereby cell lines expressing a xenotropic envelope protein block later infection by xenotropic MLV vectors able to otherwise infect those cell types, vector particles containing other viral envelopes (such as VSV-g protein (Florikiewicz, et al., J. Cell Bio. 97:1381, 1983; and Roman, et al., Exp. Cell Res. 175:376, 1988) which bind to cell receptors other than the xenotropic receptor) may be generated in the following manner. 10 μg of the plasmid DNA encoding the retroviral vector construct to be packaged is co-transfected into a cell line which expresses high levels of gag/pol with 10 μg of DNA from which a VSV-g protein is expressed. The resultant vector, containing VSV-g protein, is produced transiently in the co-transfected cells. Two days after transfection, cell free supernatants are added to prospective xenotropic packaging cell lines (which express gag, pol, and env). Cell free supernatants are then collected from the confluent monolayers and titered by PCR Cell clones producing the highest titers are selected as packaging cell lines. This procedure is sometimes referred to “G-hopping.”

VIII. Alternative Viral Vector Packaging Techniques

Several additional alternative systems can be used to produce recombinant retrovirus particles carrying a vector construct according to the invention. Some of these systems take advantage of the fact that the insect virus, baculovirus, and the mammalian viruses, vaccinia and adenovirus, have been adapted to make large amounts of any given protein for which the corresponding gene has been cloned. For example, see Smith, et al. (Mol. Cell. Biol. 3:12, 1983); Piccini, et at. (Meth. Enzymology, 153:545, 1987); and Mansour, et al. (Proc. Natl. Acad. Sci. USA 82:1359, 1985). These and similar viral vectors can be used to produce proteins in tissue culture cells by insertion of appropriate genes and, hence, could be adapted to make retroviral vector particles.

Adenovirus vectors are derived from nuclear replicating viruses and can be defective. Genes can be inserted into vectors and used to express proteins in mammalian cells either by in vitro construction (Ballay, et al., EMBO J. 4:3861, 1985) or by recombination in cells (Thummel, et al., J. Mol. Appl. Genetics 1:435, 1982).

One preferred method is to construct plasmids using the adenovirus Major Late Promoter (MLP) driving: (1) gag/pol, (2) env, (3) a modified viral vector construct. A modified viral vector construct is possible because the U3 region of the 5′ LTR, which contains the viral vector promoter, can be replaced by other promoter sequences (see, for example, Hartman, Nucl. Acids Res. 16:9345, 1988). This portion will be replaced after one round of reverse transcriptase by the U3 from the 3′ LTR.

These plasmids can then be used to make adenovirus genomes in vitro (Ballay, et al., supra), which are then transfected into 293 cells (a human cell line making adenovirus E1A protein), for which the adenoviral vectors are defective, to yield pure stocks of gag/pol, env and retroviral vector carried separately in defective adenovirus vectors. Since the titers of such vectors are typically 10⁷-10¹¹/ml, these stocks can be used to infect tissue culture cells simultaneously at high multiplicity. The cells will then be programmed to produce retroviral proteins and retroviral vector genomes at high levels. Since the adenovirus vectors are defective, no large amounts of direct cell lysis will occur and retroviral vectors can be harvested from the cell supernatants.

Other viral vectors such as those derived from unrelated retroviral vectors (e.g., RSV, MMTV or HIV) can be used in the same manner to generate vectors from primary cells. In one embodiment, these adenoviral vectors are used in conjunction with primary cells, giving rise to retroviral vector preparations from primary cells.

Another alternative for making recombinant xenotropic retroviral particles is an in vitro packaging system. For example, such a system can be employ the following components:

1. gag/pol and env proteins made in the baculovirus system in a similar manner as described in Smith, et al., supra, or in other protein production systems, such as yeast or E. coli);

2. vector constructs made using T7 or SP6 transcription systems or other suitable in vitro RNA-generating system (see, for example, Flamant and Sorge, J. Virol. 62:1827, 1988);

3. tRNA made as in (2) or purified from yeast or mammalian cells;

4. liposomes (preferably with embedded env protein); and

5. cell extract or purified components (typically from mouse cells) to provide env processing, and any or other necessary cell-derived functions.

Within this procedure, the components of (1), (2), and (3) are mixed. The env protein, cell extract and pre-liposome mix (in a suitable solvent) is then added. In a preferred embodiment, the env protein is embedded in the liposomes prior to adding the resulting liposome-embedded env to the mixture of (1), (2), and (3). The mix is treated (e.g., by sonication, temperature manipulation, or rotary dialysis) to allow encapsidation of the nascent viral particles with lipid plus embedded env protein in a manner similar to that for liposome encapsidation of pharmaceuticals, as described in Gould-Fogerite, et al., Anal. Biochem. 148:15, 1985). This procedure allows the production of high titers of replication incompetent recombinant retroviruses without contamination with pathogenic retroviruses or replication-competent retroviruses.

D. Detection of Replication Competent Retroviruses (RCR)

The propensity of the packaging cells described above to generate replication competent retrovirus may be stringently tested by a variety of methods, two of which are described below.

i. The Extended S⁺L⁻ Assay

The extended S⁻L⁻ assay determines whether replication competent, infectious virus is present in the supernatant of the cell line of interest. The assay is based on the empirical observation that infectious retroviruses generate foci on the indicator cell line MiCl₁ (ATCC No. CCL 64.1). The MiCl₁ cell line is derived from the MvlLu mink cell line (ATCC No. CCL 64) by transduction with Murine Sarcoma Virus (MSV). It is a non-producer, non-transformed, revertant clone containing a replication defective murine sarcoma provirus, S⁺, but not a replication competent murine leukemia provirus, L⁻. Infection of MiCl₁ cells with replication competent retrovirus “activates” the MSV genome to trigger “transformation” which results in foci formation.

Supernatant removed from the cell line to be tested for presence of replication competent retrovirus and passed through a 0.45 μm filter to remove any cells On day 1, Mv1Lu cells are seeded at 1.0×10⁵ cells per well (one well per sample to be tested) on a 6 well plate in 2 mL Dulbecco's Modified Eagle Medium (DMEM), 10% FBS and 8 μg/mL polybrene. MvlLu cells are plated in the same manner for positive and negative controls on separate 6 well plates. The cells are incubated overnight at 37° C., 10% CO₂. On day 2, 1.0 mL of test supernatant is added to the MvlLu cells. The negative control plates are incubated with 1.0 mL of media. The positive control consists of three dilutions (200 focus forming units (ffu), 20 ffu and 2 ffu each in 1.0 mL media) of MA virus (Miller, et al., Molec. and Cell Biol., 5:431, 1985) which is added to the cells in the positive control wells. The cells are incubated overnight On day 3, the media is aspirated and 3.0 mL of fresh DMEM and 10% FBS is added to the cells. The cells are allowed to grow to confluency and are split 1:10 on day 6 and day 10, amplifying any replication competent retrovirus. On day 13, the media on the MvlLu cells is aspirated and 2.0 mL DMEM and 10% FBS is added to the cells. In addition the MiCl₁ cells are seeded at 1.0×10⁵ cells per well in 2.0 mL DMEM, 10% FBS and 8 μg/mL polybrene. On day 14, the supernatant from the Mv1Lu cells is transferred to the corresponding well of the MiCl₁ cells and incubated overnight at 37° C., 10% CO₂. On day 15, the media is aspirated and 3.0 mL of fresh DMEM and 10% FBS is added to the cells. On day 21, the cells are examined for focus formation (appearing as clustered, refractile cells that overgrow the monolayer and remain attached) on the monolayer of cells. The test article is determined to be contaminated with replication competent retrovirus if foci appear on the MiCl₁ cells.

ii. Cocultivation of Producer Lines and MdH Marker Rescue Assay

As an alternate method to test for the presence of RCR in a retroviral particle producing cell line, producer cells are cocultivated with an equivalent number of Mus dunni cells (NIH NIAID Bethesda Md.). Small scale cocultivations are performed by mixing of 5.0×10⁵ Mus dunni cells with 5.0×10⁵ producer cells and seeding the mixture into 10 cm plates (10 mL standard culture media/plate, 4 μg/mL polybrene) at day 0. Every 3-4 days the cultures are split at a 1:10 ratio and 5.0×10⁵ Mus dunni cells are added to each culture plate to effectively dilute out the producer cell line and provide maximum amplification of RCR. On day 14, culture supernatants are harvested, passed through a 0.45 μm cellulose-acetate filter, and tested in the MdH marker rescue assay. Large scale co-cultivations are performed by seeding a mixture of 1.0×10⁸ Mus dunni cells and 1.0×10⁸ producer cells into a total of twenty T-150 flasks (30 mL standard culture media/flask, 4 μg/mL polybrene). Cultures are split at a ratio of 1:10 on days 3, 6, and 13 and at a ratio of 1:20 on day 9. On day 15, the final supernatants are harvested, filtered and a portion of each is tested in the MdH marker rescue assay.

The MdH marker rescue cell line is cloned from a pool of Mus dunni cells transduced with LHL, a retroviral vector encoding the hygromycin B resistance gene (Palmer, et al., Proc. Nat'l. Acad. Sci. USA, 84:1055, 1987). The retroviral vector can be rescued from MdH cells upon infection of the cells with RCR. One mL of test sample is added to a well of a 6-well plate containing 1×10⁵ MdH cells in 2 mL standard culture medium (DMEM with 10% FBS, 1% 200 mM L-glutamine, 1% non-essential amino acids) containing 4 μg/mL polybrene. Media is replaced after 24 hours with standard culture medium without polybrene. Two days later, the entire volume of MdH culture supernatant is passed through a 0.45 μm cellulose-acetate filter and transferred to a well of a 6-well plate containing 5.0×10⁴ Mus dunni target cells in 2 mL standard culture medium containing polybrene. After 24 hours, supernatants are replaced with standard culture media containing 250 μg/mL of hygromycin B and subsequently replaced on days 2 and 5 with media containing 200 μg/mL of hygromycin B. Colonies resistant to hygromycin B appear and are visualized on day 9 post-selection, by staining with 0.2% Coomnassie blue.

### Example 5

### Production of Recombinant Retroviral Particles

The production of recombinant retroviral particles carrying vector constructs according to the invention, representative examples of which are described above in Example __, from the human xenotropic and canine amphotropic packaging cell lines HX and DA, respectively, is described below.

A. Transient Plasmid DNA Transfection of Packaging Cell Lines HX and DA

The packaging cell line HX or DA is seeded at 5.0×10⁵ cells on a 10 cm tissue culture dish on day 1 with DMEM and 10% fetal bovine senum (FBS). On day 2, the media is replaced with 5.0 mL fresh media 4 hours prior to transfection. Standard calcium phosphate-DNA co-precipitations are performed by mixing 40.0 μl 2.5 M CaCl₂, 10 μg of the plasmid encoding the vector to be packaged, and deionized H₂O to a total volume of 400 μl. The DNA-CaCl₂ solutions are then added dropwise with constant agitation to 400 μl of precipitation buffer (50 mM HEPES-NaOH, pH 7.1; 025 M NaCl and 1.5 mM Na₂HPO₄—NaH₂PO₄). These mixtures are incubated at room temperature for 10 minutes. The resultant fine precipitates are added to different culture dishes of cells. The cells are incubated with the DNA precipitate overnight at 37° C. On day 3, the media is aspirated and fresh media is added. Supernatants are removed on day 4, passed through 0.45 μm filters, and stored at −80° C.

B. Packaging Cell Line Transduction

DA packaging cells are seeded at 1.0×10⁵ cells/3 cm tissue culture dish in 2 mL DMEM and 10% FBS, 4 μg/ml polybrene (Sigma, SL Louis, Mo.) on day 1. On day 2, 3.0 mL, 1.0 mL and 0.2 mL of each of a freshly collected supernatant containing VSV-g pseudotyped retroviral particles carrying the desired vector are added to the HX cells. The cells are incubated overnight at 37° C. On day 3, the pools of cells are cloned by limiting dilution by removing the cells from the plate and counting the cell suspension, diluting the cells suspension down to 10 cells/mL and adding 0.1 mL to each well (1 cell/well) of a 96 well plate (Corning, Corning, N.Y.). Cells are incubated for 14 days at 37° C., 10% CO₂. Several clones producing the desired recombinant xenotropic retrovirus are selected and expanded up to 24 well plates, 6 well plates, and finally to 10 cm plates, at which time the clones are assayed for expression of the appropriate retroviral vector and the supernatants are collected and assayed for retroviral titer.

The packaging cell line DA may be similarly transduced with recombinant retroviral vectors generated by G-hopping.

Using the procedures above, DA and HX cell lines may be derived that produce recombinant retroviral vectors with titers greater than or equal to 1×10⁶ cfu/mL in culture.

C. Titer Assays

Normally vector titers are determined by transduction of target cells such as HD1080, with appropriate dilutions of a vector preparation, followed by antibiotic selection and counting of surviving colonies (WO 91/02805). However, recombinant xenotropic retroviral vectors carrying a desired vector construct may not include a gene coding for a selectable marker, as may be the case when the vector construct encodes a large gene of Interest, for instance, full length factor VIII, titering assays other than those based on selection of drug resistant colonies are required. To this end, antibody and PCR assays, the latter of which is described below, may be employed to determine retroviral vector titer, i.e., the number of infectious particles comprising the retroviral vectors of the invention. While such a PCR assay may be required in the context of a vector lacking a selectable marker, it is understood that such an assay can be employed for any given vector.

To use PCR to amplify sequences unique to the retroviral vectors of the invention, various primers are required. Such primers can readily be designed by those skilled in the art and will depend on the retroviral vector backbone employed and the components thereof, the particular region(s) desired to be amplified, etc. Representative examples of particular primer pairs include those specific for LTR sequences, packaging signal sequences or other regions of the retroviral backbone, and also include primers specific for the gene of interest in the vector. Additional advantages in using such a PCR titering assay include the ability to assay for genome rearrangement, etc.

In the practice of the present invention, the PCR titering assay is performed by growing a known number of HT1080 cells, typically 1×10⁵ cells, transduced with a retroviral vector capable of directing expression of the gene of interest on 6-well plates for at least 16 hr. before harvest. The retroviral vectors used for these transductions are preferably obtained from cell culture supernatants. One well per plate is reserved for cell counting. Cells from the other wells are lysed and their contents isolated. DNA is prepared using a QIAmp Blood Kit for blood and cell culture PCR (QIAGEN, Inc., Chatsworth, Calif.). DNAs are resuspended at 5×10⁶ cell equivalents/mL, where one cell equivalent is equal to the DNA content of one cell.

To calculate titer, a standard curve is generated using DNA isolated from untransduced HT1080 cells (negative control) and HT1080 cells transduced with a known vector and having one copy of that vector per cell genome (positive control), such as may be prepared from packaging cell lines transduced with a retroviral vector encoding a selectable marker, e.g., neomycin resistance. For both the positive and negative controls, DNA is resuspended at 5×10⁶ cell equivalents/mL. The standard curve is generated by combining different amounts of the positive and negative control DNA, while keeping the total amount of DNA constant, and amplifying specific sequences therefrom by PCR using primers specific to a particular region of the retroviral vector. A representative group of mixtures for generating a standard curve is:

| Tube | 100% | 75% | 50% | 25% | 10% | 5% | 0% | Blank |
|---|---|---|---|---|---|---|---|---|
| Positive | 50 | 37.5 | 25 | 12.5 | 5 | 2.5 | 0 | 0 |
| Control (μL) | | | | | | | | |
| Negative | 0 | 12.5 | 25 | 37.5 | 45 | 47.5 | 50 | 0 |
| Control (μL) | | | | | | | | |
| Distilled water | 0 | 0 | 0 | 0 | 0 | 0 | 0 | 50 |
| (μL) | | | | | | | | |

5.0 μL from each tube is placed into one of eight reaction tubes (duplicates are also prepared), with the remainder being stored at −20° C. 5.0 μL from each sample DNA preparation are placed into their own reaction tubes in duplicate. PCR reactions (50 μL total volume) are then initiated by adding 45.0 μL of a reaction mix containing the following components per tube to be tested: 24.5 μL water, 5 μL 10×reaction PCR buffer, 4 μL of 25 mM MgCl₂, 4 μL dNTPs (containing 2.5 mM of each of dATP, dGTP, dCTP, and dTTP), 5 μL of primer mix (100 ng or each primer), 0.25 μL TaqStart monoclonal antibody (Clontech Laboratories, Inc., Palo Alto, Calif.), 1.00 μL TaqStart buffer (Clontech Labs, Inc.), and 0.25 μL AmpliTaq DNA polymerase (Perkin-Elmer, Inc., Norwalk, Conn.). Just prior to aliquoting the reaction mix to the reaction tubes, 1 μL of a³²P dCTP (250 μCi; 3000 C/mmol, 10 mCi/mL, Amersham Corp., Arlington Heights, Ill.) is added into the reacton mix. After aliquoting 45.0 μL the reaction mix into each of the reaction tubes, the tubes are capped and placed into a thermocycler. The particular denaturation, annealing, elongation times and temperatures, and number of thermocycles will vary depending on size and nucleotide composition of the primer pair used. 20 to 25 amplification thermocycles are then performed 5 μL of each reaction is then spotted on DE81 ion exchange chromatography paper (Whatman Maidstone, England) and air dried for 10 min. The filter is then washed five times, 100 mL per wash, in 50 mM Na₂PO₄, pH 7, 200 mM NaCl, after which it is air dried and then sandwiched in Saran Wrap. Quantitation is performed on a PhosphoImager SI (Molecular Dynamics, Sunnyvale, Calif.). Filters are typically exposed to a phosphor screen, which stores energy from ionizing radiation, for a suitable period typically about 120 min. After exposure, the phosphor screen is scanned whereby light is emitted in proportion to the radioactivity on the original filter. The scanning results are then downloaded and plotted on a log scale as cpm (ordinate) versus percent positive control DNA (abscissa). Titers (infectious units/mL) for each sample are calculated by multiplying the number of cells from which DNA was isolated by the percentage (converted to decimal form) determined from the standard curve based on the detected radioactivity, divided by the volume of retroviral vector used to transduce the cells. As will be appreciated by those in the art, other methods of detection, such as colorimetric methods, may be employed to label the amplified products.

### Example 6

### Large Scale Production of Recombinant Xenotropic Retroviruses

The recombinant xenotropic retroviruses of the invention can be cultivated in a variety of modes, such as in a batch or continuous mode. In addition, various cell culture technologies can be employed to produce commercial scale quantities of the recombinant xenotropic retroviruses according to the invention. Several such techniques are described below, although others known to those in the art may likewise be employed.

A. Recombinant Retrovirus Production From Hollow Fiber Cultures

i. Culture Initiation

To initiate a hollow fiber culture, the hollow fiber bioreactor (e.g. HFB; Cellco, Inc., Germantown, Md.) is first conditioned for 48 hours prior to seeding by simulating a run condition with 100-200 mL of complete growth media at 37° C. The growth media preferably is that to which the cell line has been adapted. All liquids in the HFB when originally shipped should be aspirated and replaced with the complete growth media. When seeding the bioreactor, the cells should not have been split more than 48 hours earlier and should be in log growth phase at the time of harvest for the seeding of the HFB. The cells typically are harvested by typsinazation and pelleted by centrifugation. The cell pellet is then resuspended in 4 mL of 25% preconditioned media and delivered to the extra-capillary space by syringe using the side syringe ports found on the HFB. After seeding the HFB, the cells are allowed to adhere for 20 to 30 minutes before starting the circulation pump. During this time, the media used to condition the HFB is replaced with 100-200 mL of 25% preconditioned media. The circulation feed pump is initiated with the starting flow rate set at 25 mL/min. (setting 5 with 2 long pump pins). After 1 hour from the time of switching the pump on, a one mL sample of media is collected in order to record the initial levels of lactate and ammonia. On a daily schedule, 1 ml samples are collected every 24 hours to assay for the daily production of lactate and ammonia. The initial 100-200 mL of media is exchanged with fresh media when lactate levels begin to reach 2.0 g/L (or the equivalent to 22 mM/L). The same volume of media is replaced until the culture approaches daily levels of 20 mmol/L. When daily levels of lactate reach 20 mmol/L, the size of the reservoir bottle is increased to a 500 mL bottle containing 500 mL of fresh media. The flow feed rate is then increased to 50 mL/min. when the culture begins to produce 2.2 mmol/day of lactate. When daily 500 mL volumes reach 20 mmol/L of lactate, the original Cellco supplied reservoir feeding cap is exchanged for a larger reservoir cap (Unisyn-vender part #240820) adapted for the Cellco system with the addition of tubing and male luer lock fittings. This reservoir cap will accommodate 2 liter Corning bottles. (To avoid the exchange of reservoir caps during a culture run, intiate the run with a large reservoir cap which can also support smaller bottle sizes.) When daily lactate readings are assayed and recorded, the daily levels of lactate production of the culture can be used to determine when the culture reaches maximum cell density, i.e., when the rate of lactate decreases and levels off.

ii. Seeding Density for the 2×-β-gal

To establish specific seeding requirements, two hollow fiber runs are performed, one run seeded with a low number of cells, the other seeded with a high number of cells. Progress of each culture is tracked by analyzing the daily glucose consumption and lactate production levels.

In this experiment, one HFB was seeded with 1.3×10⁷ cells (representing the low seed culture), the other with 1.6×10⁸ cells. Here, the cell line 2×-β-GAL₁₇₋₁₄ was able to initiate a good hollow fiber run under both seeding conditions. Initiating a run with fewer cells is primarily convenient for reducing the effort required for generating the number of cells required to start a culture, although fewer cells initially extends the time it takes to reach optimal cell densities, which usually yield the highest titers. 2×-β-GAL₁₇₋₁₄ adapted well to hollow fiber culture, eventually requiring daily media changes of 500 mL in order to avoid accumulation of toxic levels of lactate. Plateauing of daily lactate production and drops in peak titer production correlated with maximum cell densities and the relative health of the culture.

iii. Optimal Titer Concentrations Frequency of Harvests and Total Harvest Amounts

β-gal titers for the above experiment were determined from frozen samples on 293 cells assayed 48 or 72 hours after transduced. The transduced cells were stained for β-gal activity and counted on a hemocytometer to yield a titer based on the number of blue cells /mL (BCT/mL). Optimum tiers were generally obtained on day 7 of a high seed culture at 1.8×10⁸ BCT/mL from a 72 hour blue cell titer on 293 cells. A duplicate culture initially seeded at a 10 fold lower seeding density peaked at 5.2×10⁷ BCT/mL from a 48 hour blue cell titer. Compared to flat stock cultures (from tissue culture dishes or flasks) titered using 48 hour blue cell titers on HT1080 cells (calculated to be about 5×10⁶ BCT/mL), the increase in titer by using hollow fiber systems is approximately ten fold higher. These maximum titers observed were reached prior to hitting 20 mmol/L lactate levels, which appeared to reduce titers produced the following week.

Crude supernatants can be harvested every 9 hours with out any loss of titer and three harvests per day should be possible with minimum titre loss. In addition, continuous hollow fiber cultures can be maintained for several weeks. When titers were compared between the low and the high seed culture, there was little differences by day 11 between the two seed cultures, both of which averaged 4×10⁷ BCT/mL.

### Example 7

### Two-Phase Purification of Recombinant Retroviruses

A. Concentration of DA/ND-7 Recombinant Particles

1400 ml of media (DMEM containing 5% Fetal Bovine Serum) containing DX/ND-7 vector at a titer of 1.25×10⁶ cfu/ml is used as starting material. Three hundred milliliters of two-phase partitioning components (PEG-8000 (autoclaved), dextran-sulfate, and NaCl) are added to a final concentration of 6.5% PEG, 0.4% dextan-sulphate, and 0.3 M NaCl. The resultant solution is placed into a two-liter separatory funnel, and left in a cold room for 24 hours (including two mixing steps approximately 6 to 16 hours apart).

Following the 24 hour period, the bottom layer (approximately 20 mL) is carefully eluted, and the interphase (approximately 1 mL) is collected in a 15 mL conical FALCON tube. The interphase containing vector is diluted to 10 mL by addition of PBS, and incubated at 37° C. in order to bring the solution to room temperature and destabilize the micelles.

To one-half of the diluted interphase, KCl is added to a final concentration 0.4 M, and mixed well. The tube is then placed on ice for ten minutes, and spun for 2 minutes at 2,000 rpm in a bench-top centrifuge. The supernatant is removed and filtered through a 0.45 μm syringe filter. The other half of the interphase containing vector is separated by S-500 Sephadex chromatography in 1×PBS. The results of these concentration processes, as determined in a BCFU assay, are shown below in Table 6:

TABLE 6

| PHASE | QUANTITY OF VECTOR |
|---|---|
| Crude | 1.75 × 10⁹ bcfu |
| Separation: Top phase | 1.4 × 10⁸ bcfu |
| Separation: Interphase | 7(+/−3) × 10⁸ bcfu |
| Separation: Bottom phase | 2 × 10⁶ bcfu |
| Final step: KCl separation | *6(+/−3) × 10⁸ bcfu |
| Final step: S-500 separation | *1.8(+/−0.3) × 10⁸ bcfu |

* Note that since the sample was split into two halves, that these numbers were doubled in order to represent the level of purification that would be expected if the entire 1 mL interphase was separated as indicated.

In summary, 1.4 liters of crude research grade supernatant containing recombinant retroviral particles may be reduced to a 10 mL volume, with approximately 50% (+/−20%) being recovered when KCl separation is utiized as the final step. When S-500 chromatography is as the final step, only about 10% of the initial recombinant retroviral particles are recovered in a 14 mL.

In order to complete concentration of the retroviral vector particles, the vector-containing solution may be further subjected to concentration utilizing an MY-membrane Amicon filter, thereby reducing the volume from 10 to 14 mL, down to less than 1 mL.

### Example 8

### Production Vector From DX/ND7 B-Gal Clone 87 Utilizing a Cell Factory

DX/ND7 bgal clone 87, an expression vector, was grown in cell factories. Cells were grown in DMEM supplemented with Fetal Bovine Serum in roller bottles until enough cells to seed 20 10-layer cell factories (NUNC) at a 1:3 dilution were obtained Each 10-layer cell factory is seeded with approximately 0.8 liters of cell medium.

Cells were seeded into the cell factory by pouring media containing cells into the factory so that the suspensions evenly fill the 10 layers. The factory is then carefully tilted away from the port side to prevent the suspension from redistribution in the common tube. Finally, the cell factory is rotated into its final upright position. A hepavent filter is attached to each port. The factory was then placed in a CO₂ incubator.

In three days, and for each of the next three days, supernatant containing vector was harvest. The cell factory is placed in a tissue culture hood. One filter is removed and sterile transfer tubing is connected to the open port. The factory is lifted so that supernatant drams into the tubing. Approximately 2 liters of supernatant is harvested from each factory. Fresh DMEM/FBS is used to replenish the lost medium. The transfer tubing is removed and the factory replaced in the incubator. From 20 cell factories, approximately 90 liters of crude vector containing supernatant were obtained.

Verification of the vector was performed by transduction of HT1080 cells. These cells were harvested 2 days law and stained for b-gal protein. The titer of the supernatant was determined to be 2×10⁷/mL

### Example 9

### Concentration of Recombinant Retrovirus by Low-Speed Centrifugation

A. Retrovector Supernatant Preparation

Producer cell lines DA/βgal and HX/DN-7 were cultured in a culture flask and a roller bottle, respectively, containing Dulbecco's Modified Eagle's Medium (DMEM) supplemented with 10% fetal bovine serum plus 1 mM L-Glutamine, Sodium pyruvate, non-essential amino acids and antibiotics. Viral supernatant was harvested from the flask and roller bottle, and were filtered through a 0.4 um syringe filter. The filtered supernatants were stored either at 4° C. (HX/ND7), or frozen at −70° C. (DAβ-gal).

B. Virus Concentration

Viral supernatant was aliquoted into 50 ml sterile OAKRIDGE screw cap tubes, and placed into an SS34 rotor for use in a Sorvall centrifuge. The tubes were spun for 1 hour at 16,000 rpm (25,000 g-force) at 4° C. Upon completion of the spin, the tubes were removed, the supernatant decanted and a small opaque pellet resuspended in the DMEM media described above.

C. Virus Titration

Concentrated virus was titered on HT1080 cells plated 24 hours earlier at a cell density of 2×10⁵ cells per well in a six well plate+4 μg/ml polybrene. Briefly, virus preps were diluted from 1/10 to 1/10,000 and 50 μl of each dilution was used to infect one well from the six well plate. Plates were incubated overnight at 37° C. Fort-eight hours later, cells were fixed and stained with X-gal. The results are set forth below in Table 7.

TABLE 7
Virus Concentration through Low Speed Centrifugation

| | Experiment number | | | | |
|---|---|---|---|---|---|
| Parameter description | 1 | 2 | | 3 | |
| Virus source | DAβ-gal | DAβ-gal | HX/ND7 | DAβ-gal | HX/ND7 |
| Titer of normal harvest | 4.4 × 10⁶ | 2.1 × 10⁶ | 3.2 × 10⁵ | 5 × 106 | 5 × 105 |
| Titer of virus concentrate | 6 × 10⁸ | 7.4 × 10⁷ | 3.2 × 10⁷ | 2.9 × 108 | 3.9 × 107 |
| Starting volume | 80 ml | .39 ml | 39 ml | 118 ml | 40 ml |
| Final concentrate volume | .5 ml | .36 ml | .36 ml | .78 ml | .28 ml |
| Fold virus concentration | 136X | 34X | 100X | 58X | 78X |
| Virus recovery | 87% | 30% | 91% | 50% | 99% |

As is evident from Table 7, virus recovery ranged from 30% to 99%, with the best recovery being obtained from human producer cells (HX/ND7; recovery ranged from 91% to 99%).

### Example 10

### Concentration of Recombinant Retroviruses by Ultrafiltration

S-500 purified supernatant containing the β-gal expressing recombinant retrovirus DX/CB-bgal and partially concentrated supernatant containing the same virus were each filtered through a 0.45 um filter, and loaded into a CENTRIPREP-100 filter (product #4308, Amicon, Mass.). The supernatants were kept at a temperature of 4° C. throughout this procedure, including during centrifugation. The CENTRIPREP filters were spun three times each for 45 to 60 minutes at 500×G. Between each spin the filtrate was decanted. The retentate was thus sequentially reduced, such that the initial 15 mL (or 10 mL) volume was reduced to approximately 0.6 mL per unit.

The resultant titer was determined by assaying HT1080 target cells set up at a concentration of 1×10⁵ cells per well 24 hours prior to transduction of the viral sample. Cells were transduced in the presence of 8 μg/ml polybrene and 2 mL growth media (DMEM plus 10% FBS) per well. As shown in Table 8 below, approximately one hundred percent of the virus was recovered utilizing this procedure (note that titers are in BCFU/ml).

TABLE 8

| | Pre-centriprep | Final |
|---|---|---|
| | titer/volume. | titer/volume |
| S-500 | 4 × 10⁷/15 ml | 1.3 × 10⁹/0.6 ml |
| part. conc. | 3 × 10⁸/10 ml | 1 × 10¹⁰/0.6 ml |

### Example 11

### Preparation of Recombinant Retrovirus in a Bioreactor

A. Freezing Protocol

Producer cells are frozen in DMEM media containing 10% to 20% FBS, and 5 to 15% DMSO, at a concentration of 1×10⁷ cells/vial. Cells are frozen in a controlled rate freezer (Series PC, Controlled Rate Freeing System, Custom Biogenic Systems, Warren Mich.) at a rate of from 1 to 10° C. per minute. Frozen cells are stored in liquid nitrogen.

B. Biorector Protocol

Cells are thawed from frozen vials at 37° C., washed once with media to remove DMSO, and expanded into 850 cm² “FALCON” roller bottles (Corning, Corning, N.Y.) Expanded cell culture is used to inoculate a “CELLIGEN PLUS” bioreactor (5 liter working volume; New Brunswick, Edison, N.J.). The cells are grown on microcarriers (i.e., Cytodex 1 or Cytodex 2; Pharmacia, Piscataway, N.J.) at a concentration of 3 to 15 g/L microcarrier. Initial inoculation densities are from 4 to 9 cells/bead at half to full volume for 2 to 24 hours. The media constituents for virus production are DMEM-high glucose (Irvine Scientific, Santa Ana, Calif.) basal media supplemented with FBS (10 to 20%), Glutamine (8 to 15 mM), glucose (4.5 to 6.5 g/L), Nonessential amino acids (1×), RPMI 1640 amino acids (0.2 to 9.6×), 10 mM HEPES, RPMI 1640 Vitamins (0.2 to 5×).

During culture, pH (6.9 to 7.6) and dissolved oxygen (“DO” 5 to 90%) are controlled by the use of a four gas system which includes air, oxygen, nitrogen, and carbon dioxide. After several days of batch growth the culture is then continuously perfused with fresh media with concurrent continuous harvesting in an escalating perfusion rate of 0.5 to 2.5 volumes/day. Cell retention is the result of differential sedimentation of cell covered beads in a decanting column.

During operation the bioreactor is monitored for viable cells, titer, glucose, lactate, ammonia levels, and lack of contamination. Viable cells and titer range from 1×10⁵ cells/ml to 1×10⁷ cells/ml. Glucose ranges from 6 to 0.25 g/L, Lactate from 1 to 25 mM, and Ammonia ranges from 0.5 to 30 mM. Cells are incubated in the bioreactor for 5 to 25 days.

While the present invention has been described above both generally and in terms of preferred embodiments, it is understood that variations and modifications will occur to those skilled in the art in light of the description supra. Therefore, it is intended that the appended claims cover all such variations coming within the scope of the invention as claimed.

Additionally, the publications and other materials cited to illuminate the background of the invention, and in particular, to provide additional details concerning its practice as described in the detailed description and examples, are hereby incorporated by reference in their entirety.

We claim:

1. A method of producing transduced mammalian T cells or non-dividing cells, the method comprising:
(a) obtaining a population of T cells or non-dividing cells from a patient; and
(b) transducing the population of T cells or non-dividing cells ex vivo with a preparation of high titer recombinant retroviral particles substantially free from contamination with replication competent retrovirus, wherein the recombinant retroviral particles carry a vector construct encoding a gene of interest.

2. The method of claim 1 wherein said T cells are isolated CD4+ T cells.

3. The method of claim 1 wherein said T cells are isolated CD8+ T cells.

4. The method of claim 1 wherein the gene of interest encodes a protein or active portion of a protein selected from the group consisting of a cytokine, a colony stimulating factor, a clotting factor, and a hormone.

5. The method of claim 4 wherein said clotting factor is factor VIII.

6. The method of claim 1 wherein the patient is a human suffering from a disease selected from the group consisting of a genetic disease, a cancer, an infectious disease, an autoimmune disease, a cardiovascular disease, degenerative disease, and an inflammatory disease.

7. A composition comprising an isolated population of mammalian T cells or non-dividing cells, transduced ex vivo with a preparation of high titer recombinant retroviral particles substantially free from contamination with replication competent retrovirus, wherein the recombinant particles carry a vector construct encoding a gene of interest.

8. The composition of claim 7 wherein said T cells are isolated CD4+ T cells.

9. The composition of claim 7 wherein said T cells are isolated CD8+ T cells.

10. The composition of claim 7 wherein the gene of interest encodes a protein or active portion of a protein selected from the group consisting of a cytokine, a colony stimulating factor, a clotting factor, and a hormone.

11. The composition of claim 10 wherein said clotting factor is factor VIII.

12. The composition of claim 7 wherein said mammalian cells are human cells.

13. A mammalian T cell or non-dividing cell transduced ex vivo with a preparation of high titer recombinant retroviral particles substantially free from contamination with replication competent retrovirus, wherein the recombinant retroviral particles carry a vector construct encoding a gene of interest.

14. The T cell of claim 13 wherein said T cell is from an isolated population of CD4+ T cells.

15. The T cell of claim 13 wherein said T cell is from an isolated population of CD8+ T cells.

16. The T cell or non-dividing cell of claim 13 wherein the gene of interest encodes a protein or active portion of a protein selected from the group consisting of a cytokine, a colony stimulating actor, a clotting actor, and a hormone.

17. The T cell or non-dividing cell of claim 16 wherein the clotting factor is factor VIII.

18. A method of ting a patient having a genetic disease, the method comprising:
(a) obtaining a population of T cells or non-dividing cells from the patient;
(b) transducing the population of T cells or non-dividing cells ex vivo with a preparation of high titer recombinant retroviral particles substantially free from contamination with replication competent retrovirus, wherein the recombinant retroviral particles carry a vector construct encoding a gene of interest useful in treating the genetic disease; and
(c) re-introducing into the patient a therapeutically effective amount of the population of transduced T cells or non-dividing cells.

19. The method of claim 18 wherein said cell population is a T cell population, wherein said disease is ADA deficiency, and wherein said gene of interest is ADA.

20. The method of claim 18 further comprising expanding the transduced population of T cells, non-dividing cells prior to re-introduction of the cells into the patient.

21. A method of treating a patient having cancer, the method comprising:
(a) obtaining a population of T cells or non-dividing cells from the patient;
(b) transducing the population of T cells or non-dividing cells ex vivo with a preparation of high titer recombinant retroviral particles substantially free from contamination with replication competent retrovirus, wherein the recombinant retroviral particles carry a vector construct encoding a gene of interest useful in treating cancer; and
(c) re-introducing into the patient a therapeutically effective amount of the population of transduced T cells or non-dividing cells.

22. A method of treating a patient having an infectious disease, the method comprising:
(a) obtaining a population of T cells or non-dividing cells from the patient;
(b) transducing the population of T cells or non-dividing cells ex vivo with a preparation of high titer recombinant retroviral particles substantially free from contamination with replication competent retrovirus, wherein the recombinant retroviral particles carry a vector construct encoding a gene of interest useful in treating the infectious disease; and
(c) re-introducing into the patient a therapeutically effective amount of the population of transduced T cells or non-dividing cells.

23. The method of claim 22 wherein said cell population is a T cell population, wherein said infectious disease is AIDS, and wherein said gene of interest encodes a mutant HIV protein.

24. The method of claim 22 wherein said cell population is a T cell population, wherein said infectious disease is AIDS, and wherein said gene of interest encodes a ribozyme.

25. The method of claim 22 wherein said cell population is a T cell population, wherein said infectious disease is AIDS, and wherein said gene of interest encodes a synthetic or naturally occurring T cell receptor.

26. A method of treating a patient having an inflammatory disease, the method comprising:
(a) obtaining a population of T cells or non-dividing cells from the patient;
(b) transducing the population of T cells or non-dividing cells ex vivo with a preparation of high titer recombinant retroviral particles substantially free from contamination with replication competent retrovirus, wherein the recombinant retroviral particles carry a vector construct encoding a gene of interest useful in treating the inflammatory disease; and
(c) re-introducing into the patient a therapeutically effective amount of the population of transduced T cells or non-dividing cells.

27. A method of treating a patient having a degenerative disease, the method comprising:
(a) obtaining a population of T cells or non-dividing cells from the patient;
(b) transducing the population of T cells or non-dividing cells ex vivo with a preparation of high titer recombinant retroviral particles substantially free from contamination with replication competent retrovirus, wherein the recombinant retroviral particles carry a vector construct encoding a gene of interest useful in treating the inflammatory disease; and
(c) re-introducing into the patient a therapeutically effective amount of the population of transduced T cells or non-dividing cells.

28. A method of treating a patient having a cardiovascular disease, the method comprising:
(a) obtaining a population of T cells or non-dividing cells from the patient;
(b) transducing the population of T cells or non-dividing cells ex vivo with a preparation of high titer recombinant retroviral particles substantially free from contamination with replication competent retrovirus, wherein the recombinant retroviral particles carry a vector construct encoding a gene of interest useful in treating the cardiovascular disease; and
(c) re-introducing into the patient a therapeutically effective amount of the population of transduced T cells or non-dividing cells.

29. The method of claim 26 wherein said cell population is a T cell population, wherein said cardiovascular disease is hyperlipidemia, and wherein said gene of interest encodes apolipoprotein E.

30. A method of treating a patient having an autoimmune disease, the method comprising:
(a) obtaining a population of T cells or non-dividing cells from the patient;
(b) transducing the population of T cells or non-dividing cells ex vivo with a preparation of high titer recombinant retroviral particles substantially free from contamination with replication competent retrovirus, wherein the recombinant retroviral particles carry a vector construct encoding a gene of interest useful in treating the autoimmune disease; and
(c) re-introducing into the patient a therapeutically effective amount of the population of transduced T cells or non-dividing cells.

31. A method of modulating the activity of a population of T cells or non-dividing cells in a patient comprising:
(a) obtaining the population of T cells or non-dividing cells from the patient;
(b) transducing said population of cells ex vivo with a preparation of high titer recombinant retroviral particles substantially free from contamination with replication competent retrovirus, wherein the recombinant retroviral particles carry a vector construct encoding a protein capable of activating a prodrug;
(c) re-introducing said population of cells into the patient; and
(c) administering said prodrug to said patient.

32. The method of claim 31 wherein said protein is thymidine kinase.

33. A method according to claim 1 wherein an envelope protein of the high titer recombinant retroviral particles is an envelope protein derived from a type C retrovirus or from a type D retrovirus.

34. A method according to claim 1 wherein an envelope protein of the high titer recombinant retroviral particles is an envelope protein is selected from the group consisting of a retroviral amphotropic envelope protein, a retroviral ecotropic envelope protein, a retroviral polytropic envelope protein, a retroviral xenotropic envelope protein, a gibbon ape leukemia virus envelope protein, and a VSV-g protein.


SEQUENCE LISTING (10 sequences)

SEQ ID NO: 1 - DNA, 21 bp, Artificial Sequence
  cDNA
taataaatagatttagattta

SEQ ID NO: 2 - DNA, 35 bp, Artificial Sequence
  cDNA
gcctcgagacgatgaaatatacaagttatatcttg

SEQ ID NO: 3 - DNA, 35 bp, Artificial Sequence
  cDNA
gaatcgatccattactgggatgctcttcgacctgg

SEQ ID NO: 4 - DNA, 40 bp, Artificial Sequence
  cDNA
gcctcgagctcgagcgatgaaatatacaagttatatcttg

SEQ ID NO: 5 - DNA, 27 bp, Artificial Sequence
  cDNA
gtcatctcgtttctttttgttgctatt

SEQ ID NO: 6 - DNA, 27 bp, Artificial Sequence
  cDNA
aatagcaacaaaaagaaacgagatgac

SEQ ID NO: 7 - DNA, 40 bp, Artificial Sequence
  cDNA
gcatcgatatcgatcattactgggatgctcttcgacctcg

SEQ ID NO: 8 - DNA, 21 bp, Artificial Sequence
  cDNA
ataaatagaaggcctgatatg

SEQ ID NO: 9 - DNA, 35 bp, Artificial Sequence
  cDNA
gcctcgagacaatgtacaggatgcaactcctgtct

SEQ ID NO: 10 - DNA, 35 bp, Artificial Sequence
  cDNA
gaatcgatttatcaagtcagtgttgagatgatgct